(19) United States
(12) Patent Application Publication
(10) Pub. No.: US 2004/0266834 A1
(43) Pub. Date: Dec. 30, 2004

(54) Thiazolidinediones alone or in cabination with other therapeutic agents for cancer therapy

(76) Inventors: John A. Copland III, Houston, TX (US); Slavisa Gasic, Buffalo, NY (US); Randall J. Urban, Friendswood, TX (US); Melvyn Soloff, Galveston, TX (US)

Correspondence Address: FULBRIGHT & JAWORSKI L.L.P., 600 CONGRESS AVE., SUITE 2400, AUSTIN, TX, 78701, US

(21) Appl. No.: 10/831,774

(22) Filed: Apr. 23, 2004

Related U.S. Application Data
(63) Continuation of application No. 09/418,095, filed on Oct. 14, 1999, now abandoned.

(51) Int. Cl. A61K 31/4439; A61K 31/426; A61N 5/00

(52) U.S. Cl. 514/342; 514/369; 600/1

(57) ABSTRACT

The present inventions relate to the use of thiazolidinedione compounds, including troglitazone, either alone or in combination with other cancer treatments, for the treatment of cancer. These combination therapies employ thiazolidinedione compounds in conjunction with anticancer treatments, such as chemotherapeutic agents.

This is a continuing application of United States Patent application entitled “Thiazolidinediones Alone or in Combination with Other Therapeutic Agents for Cancer Therapy,” filed on Sep. 29, 1999 on behalf of John A. Copland, III, Slavisa Gasic, Randall J. Urban, and Melvyn Soloff, which is a continuation application of U.S. Ser. No. 60/102,217, filed on Sep. 29, 1998, which is now abandoned. The entire text of the above-referenced disclosures are specifically incorporated by reference herein without disclaimer.

### BACKGROUND OF THE INVENTION

I. Field of the Invention

The present invention relates generally to the field of cancer therapeutics. More particularly, it concerns the use of thiazolidinediones, including troglitazone, by itself and in combination with chemotherapeutics in the treatment of human pre-cancers and cancers, especially osteosarcomas, tumors derived from the same precursor cell type as osteosarcomas, renal cell carcinomas and ovarian cancer.

II. Description of Related Art

In 1999, more than half a million Americans will probably die of cancer. Greater than five million people have already succumbed to the disease this decade. Surpassed only by heart disease, cancer is the second leading cause of death in the United States, with one in four deaths being the result of cancer. About 1.2 million people are expected to be diagnosed with cancer in 1999.

Normal cells are subjected to a variety of control mechanisms to control their growth and differentiation. Cancer cells, however, are cells whose growth is not controlled, as they continue to grow and divide, and possibly spread to other parts of the body. Tumors are lumps of aggregated cancer cells, which can ultimately kill normal cells and tissue. Furthermore, tumor cells can metastasize by splitting apart from the tumor and settling into another part of the body via the blood stream or the lymph system. If cells break away from such a tumor, they can travel through the bloodstream, or the lymph system to other areas of the body.

Some types of cancer, however, do not typically involve a tumor. Leukemia is an example of this type. Instead, these cancer cells involve the blood and blood-forming organs (bone marrow, lymphatic system, and spleen), and circulate through other tissues where they can accumulate. Moreover, not all tumors are cancerous. Some tumors (noncancerous) do not metastasize and are not life-threatening.

Ovarian cancer is the fourth leading cause of death from cancers among American women after lung, breast, and colon cancers. Over 90 percent of ovarian cancers are of ovarian surface epithelial origin and it is the most lethal of the gynecological malignancies due to late stage diagnosis. The overall 5-year survival rate is approximately 30% (Piver et al., 1992). Lack of symptoms and sensitive tests for detection of early stages of this disease results in over 70% of ovarian cancer patients being diagnosed at Stage III or IV classification at which time metastasis to the pelvic and abdominal viscera or distant metastasis has occurred. Current treatments for advanced stage ovarian cancer include cytoreductive surgery followed by combination chemotherapy with paclitaxel and a platinum drug e.g., cisplatin or carboplatin. Initial response rates of up to 80% are observed but patients become refractory to this therapy as well as other drugs to which they are exposed (Qazi et al., 1995). Thus, multidrug resistance (MDR) is a major clinical problem in ovarian cancer as well as other cancers.

Common ovarian epithelial cancers develop from ovarian surface epithelium of the ovary. Embryonically, ovarian surface epithelium originate from coelomic epithelium which overlies the gonadal ridge. They are of mesodermal origin and developmentally are closely related to the underlying stromal fibroblasts. Consequently, embryonic coelomic epithelium is competent to develop along multiple pathways and this capacity contributes to the variety of phenotypes found among ovarian surface epithelial-derived carcinomas. The pluripotent potential of ovarian surface epithelium in adult women is further emphasized by culturing ovarian surface epithelium from women with strong family history of breast or ovarian cancer. In culture, normal ovarian surface epithelium undergo conversion from epithelial to mesenchymal phenotype (Auersperg et al., 1994). Cultured cells from women with predisposition to ovarian cancer mimic those of ovarian carcinoma cells retaining epithelial phenotype throughout culture (Auersperg et al., 1995; Dyck et al., 1996). Since ovarian cancer is of unifocal origin (Jacobs et al., 1992; Mok et al., 1993; Tsao et al., 1993) then early stages of the disease could be detected if specific early stage markers can be identified. One described tumor suppressor gene is DOC-2, which is expressed in normal surface epithelial ovarian cells but not ovarian cancer cell lines and tissues (Mok et al., 1998). This result may hold promise for early diagnosis in the future but still does not address the current issue of treating late stage ovarian cancer.

Effective therapy for patients with metastatic renal cell carcinoma is not presently available. Current therapeutic approaches involve surgery and various types of immunotherapy. The cytokines interleukin-2 (IL-2, aldesleukin [Proleukin], interferon-alfa (Intron A, Roferon-A), or the combination produce responses in 15% to 20% of patients. Randomized trials suggest that administration of interferon-alpha may result in a modest improvement in median survival. This response rate is of short duration.

Troglitazone is a member of the thiazolidinedione class of drugs that act as ligands to activate the transcriptional factor, PPAR-γ. Ligands of PPAR-γ have previously been shown to cause in vitro terminal differentiation in human tumors of liposarcoma cells (Tontonoz et al., 1997), breast (Mueller et al., 1998), prostate (Kubota et al., 1997), and colon (Sarraf et al. 1998). Another example of differentiation therapy is the use of all-trans retinoic acid in the treatment of myelocytic lineage derived cancers such as acute promyelocytic leukemia (Warrel et al., 1993). Induction of terminal differentiation of malignant cells is an ideal mechanism of curing cancer as opposed to a cell death mediated mechanisms.

PPAR-γ plays a central role in the process of adipocyte differentiation and is expressed in high levels in this tissue. Mechanistically, PPAR-γ heterodimerizes with retinoid X receptor (RXR-α) to form a DNA-binding complex (DR-1) that regulates expression of adipocyte-specific genes (Kliewer et al., 1992; Tontonoz et al., 1994a; Tontonoz et al., 1994b; Tontonoz et al., 1995; Sears et al., 1996). The combination of PPAR-γ ligand (pioglitazone) and RXR ligand (LG268) treatment of liposarcoma cells in vitro result in an additive effect upon terminal differentiation as characterized by accumulation of intracellular lipid, induction of adipocyte-specific genes, and withdrawal from the cell cycle (Tontonoz, 1997). To date, other tissues expressing PPAR-Y include muscle, kidney, liver, and lung, (Tontonoz, 1997).

Besides its ability to bind to PPAR-γ, troglitazone is an antioxidant due to the vitamin E moiety in its structure. Vitamin E has recently been shown to induce apoptosis in human colorectal cancer cell lines (Chinery et al., 1997). However, the IC₅₀ dose of vitamin E for HCT 15 cells was 0.75 mM and for HCT 116 cells was 3 mM. Combinatorial therapy with 5-FU and 3 mM vitamin E reduces the IC₅₀ of 5-FU alone by 2- and 8-fold respectively in HCT 116 and HCT15 cells. The authors demonstrated an increase in p21 expression and conclude that vitamin E's inhibitory activity is due to the increase in the cell cycle inhibitor, p21.

Saos-2 cells are an osteogenic sarcoma characterized by a mutant nonfunctional retinoblastoma (Rb) protein and no p53 protein expression. Both of these proteins play a critical role in regulating cell cycle progression. Loss of function of these two proteins is a partial explanation for the developed resistance to chemotherapeutic agents used to treat patients. The retinoblastoma gene product (pRb) is a critical substrate of the evolutionarily conserved cyclin-dependent kinases (CDK's) that regulate progression through the cell cycle. Progression through the cell cycle is governed by a family of cyclin dependent kinases (CDKs), whose activity is governed by phosphorylation activated by binding of cyclins, and inhibited by the cdk inhibitors (reviewed in Sherr, 1996). The CDKs regulate biochemical pathways or checkpoints that integrate mitogenic and growth inhibitory signals, monitor chromosome integrity, and coordinate cell cycle transitions. Passage through G1 and S phase is regulated by the activities of cyclin E/CDK2, cyclin D/CDK4, and cyclin D/CDK6. Cyclin A/CDK2 promotes passage through the S phase and cyclin B/CDK2 kinase is essential for G2/mitosis transition. Two families of CDK inhibitors (CKIs) mediate cell cycle arrest following growth inhibitory stimuli. The INK4 family members p15INK4b/MTS2, p16INK4/MTS1, p18, and p19 bind CDK4 and CDK6 specifically and inhibit cyclin D binding. Members of the KIP family are currently composed of p21K1P1/WAF1/SDII (transcriptional target of p53), p27K1P1, p57K1P2 (reviewed in Harper et al.). These molecules specifically target CDKs that are important to G1-S transition: CDK2, CDK4, and CDK6. Thus, CKIs bind CDKs inhibiting progression through the cell cycle. Phosphorylation of CKIs leads to degradation of CKI and to cell cycle progression. Conversely loss of expression of key CKI(s) can lead to continual cell cycle progression. Compounds that can increase CKI levels are potential chemotherapeutic agents against cancer.

### SUMMARY OF THE INVENTION

The present invention addresses the need for improved cancer treatments, particularly, the need to increase the efficacy and toxicity of cancer therapies, including chemotherapeutic drug regimens, and to reduce the incidence of MDR. It is thus an object of the present invention to provide a method of treating cancer through the use of troglitazone or other thiazolidinedione compounds either alone or in combination with other chemotherapeutic agents.

The present invention relates to the use of thiazolidinedione compounds, alone or in combination with other therapeutic agents and drugs for the treatment of cancer. Treatment of cancer includes the extension of the subject's life by any period of time, decrease or delay in the neoplastic development of the disease, inhibition of cancer cell growth (including inhibition of tumor cell growth), decrease in growth or proliferation of cancer cells, reduction in tumor growth, delay or prevention of metastases, reduction in cancer cell or tumor cell proliferation rate, and a decrease in pain to the subject that can be attributed to the subject's condition.

The invention concerns any thiazolidinedione compound, including troglitazone, pioglitazone, and rosiglitazone, in the treatment of cancer. It also includes the use of combination treatments with one or more thiazolidinedione compounds and one or more other anticancer agent such as chemotherapy drugs, immunotherapy, and radiation therapy.

In some embodiments of the preferred invention, the methods concern inhibiting growth of a cancer cell comprising contacting the cancer cell with a thiazolidinedione compound in an amount effective to inhibit growth of the cancer cell. In some embodiments of the present invention, the lowest amount effective to inhibit growth of the cancer cell is preferred. As previously mentioned the invention concerns the use of thiazolidinedione compounds, including implementation of a method of inhibiting the growth of a cancer cell by contacting the cells at least with troglitazone. In another aspect of the invention, the growth of a cancer cell is inhibited by at least contacting the cell with the thiazolidinedione compounds pioglitazone or rosiglitazone. It is further contemplated that more than one thiazolidinedione compound can be used by themselves or in combination with chemotherapeutic drugs and other anticancer therapies as combination treatments or therapies.

In another embodiment, a method of the claimed invention includes inhibiting the growth of the cell by inhibition of cell cycle progression.

In some aspects, the methods of inhibiting the growth of cancer cell concern a mammalian cancer cell. In another aspect, the cancer cell is a human cell. However, any cell type is within the scope of the invention. Furthermore, it is contemplated that inhibiting the growth of a cancer cell may occur at least by either in vitro or in vivo contacting of the cancer cell.

The present methods are contemplated to cover the treatment of various cancer cells, including: bladder cell, blood cell, bone cell, bone marrow cell, brain cell, breast cell, colon cell, esophagus cell, gastrointestine cell, head cell, kidney cell, liver cell, lung cell, nasopharynx cell, neck cell, ovary cell, prostate cell, skin cell, stomach cell, and uterus cell. The type of cell is not limited, however, to these organ or tissues, and it is contemplated that the cancer treatments of the present invention can be used with other cell types as well, including cells sharing part or all of their lineage with the cell types listed above, but at a different stage of differentiation. For example, it is contemplated that a myeloid cell is included in the category of blood cell. In specific embodiments of the invention, the methods involve inhibiting the growth of a bone cancer cell, an osteosarcoma cell, a precursor cell to osteosarcoma, an ovarian cancer cell, a renal cancer cell, or any cell that expresses PPAR-γ.

In particular aspects of the claimed invention, treatment methods include contacting a cell with at least one chemotherapeutic drug in addition to at least one thiazolidinedione compound. The chemotherapeutic drugs may be, for example, selected from the group consisting of an alkylating agent, mitotic inhibitor, antibiotic, nitrosurea, antimetabolite, corticosteroid hormone, and/or other antineoplastic agent.

In yet further embodiments the chemotherapeutic drug can comprise an alkylating agent. Examples of alkylating agents that anticipated to be of use in the invention include, but are not limited to, mechlorethamine, cyclophosphamide, ifosfamide chlorambucil, melphalan, busulfan, cisplatin, dacarbazine, thiotepa, carmustine, lomustine, and shreptozoin. In other aspects of the claimed invention, cancer treatments that involve contacting a cancer cell with a thiazolidinedione compound also include contacting a cancer cell with a chemotherapeutic drug that comprises a mitotic inhibitor. Examples of mitotic inhibitors that are anticipated to be of use in the invention include, but are not limited to, vincristine, vinblastine, paclitaxel, docetaxel, etoposide, vinorelbine, and taxol. Other embodiments of the claim invention involve a chemotherapeutic drug that comprises an antibiotic. Examples of antibiotics that are anticipated to be of use in the invention include, but are not limited to, dactinomycin, daunorubicin, idarubicin, bleomycin, mitomycin, and doxorubicin. The present invention is directed also to the use of a chemotherapeutic drug that comprises a nitrosurea in the combination therapies for the treatment of cancer. Examples of nitrosureas that are anticipated to be of use in the invention include, but are not limited to, carmustine and lomustine. Furthermore, the claimed invention covers a chemotherapeutic drug that comprises an antimetabolite in the combination therapies. Examples of antimetabolites that are anticipated to be of use in the invention include, but are not limited to, 5-fluorouracil, Ara-C, fludarabine, gemcitabine, and methotrexate. In other aspects of the claimed invention, the chemotherapeutic drug comprises a corticosteroid hormone. Examples of antibiotics that are anticipated to be of use in the invention include, but are not limited to, dexamethasone and prednisone, and other such hormones are included within the scope of the combination therapies of this invention. In additional examples of the present invention, the combination treatments involve a thiazolidinedione compound with a chemotherapeutic drug that comprises an antineoplastic agent. Examples of antineoplastic agents that are anticipated to be of use in the invention include, but are not limited to, L-asparaginase, amsacrine, tretinoin, and TNF.

The methods of the present invention involve contacting a cancer cell with a thiazolidinedione compound alone or in combination with a chemotherapeutic drug, all which may be administered to a cell regionally, endoscopically, intravenously, intralesionally, percutaneously, subcutaneously, intraperitoneally, intratracheally, intramuscularly, or by perfusion.

The invention is also contemplated for use in the treatment of cancer of thiazolidinedione and the chemotherapeutic drugs that are suitably dispersed in a pharmacologically acceptable formulation.

In the combination therapies and treatments of the claimed invention covering both thiazolidinedione compounds and chemotherapeutic agents, in some embodiments of the invention, the thiazolidinedione compound is contacted with the cell prior to contact with the chemotherapeutic agent, after contact with the chemotherapeutic agent, or at the same time as contact with the chemotherapeutic agent. In some cases of the invention, the methods concern a cancer cell that is a tumor cell in a tumor. As such, some examples of the invention include a treatment further comprising resecting the tumor.

Other combination cancer treatments of the present invention involve contacting a cancer cell with a thiazolidinedione compound and further comprise irradiating said tumor cell with X-ray irradiation, UV-irradiation, γ-irradiation, or microwaves. The combination of treating with a thiazolidinedione compound and other anticancer drugs covers radiotherapy. In these cases, the thiazolidinedione compound can be contacted with the cell prior to irradiation, after irradiation, or at the same time as irradiation.

Instead of chemotherapy and radiotherapy, it is contemplated that treatment with thiazolidinedione compounds may be combined with gene therapy, which may involve contacting the cell with a therapeutic polynucleotide selected from the group consisting of a Dp gene, p21, p16, p53, FHIT, p27, E2F, Rb, APC, DC, NF-1, NF-2, WT-1, MEN-I, MEN-II, BRCA1, VHL, FCC, MCC, ras, myc, neu, raf erb, src, fms, jun, trk, ret, gsp, hst, bcl, abl, Bax, Bcl-X, and E1A.

It is contemplated that the combination treatments involving thiazolidinedione compounds and other anticancer agents may be administered individually or collectively in amounts effective to inhibit cancer in a cancer patient. Furthermore, the invention includes inhibiting the cell cycle progression of a mammalian cancer cell comprising contacting the cell with an amount of troglitazone effective to inhibit the cell cycle progression of the cell. In other embodiments the invention covers inhibiting the cell cycle progression of a mammalian cancer cell comprising contacting the cell with an amount of troglitazone effective to inhibit the cell cycle progression of the cell. In addition to troglitazone, an amount of a chemotherapeutic agent effective to inhibit the cell cycle progression of the cell is considered within the scope of the invention. In some embodiments of the invention, troglitazone lowers the amount of an anticancer drug, such as a chemotherapeutic, effective to treat cancer or inhibit growth of a cancer cell relative to the amount necessary for such a result in the absence of troglitazone treatment. Therefore, the characterization “therapeutically effective” can apply both to a combination effect of a combined therapy and to the effect of a drug individually.

Methods of the present invention also cover treating microscopic residual cancer comprising the steps of: (i) identifying a patient having a resectable tumor; (ii) resecting said tumor; and (iii) contacting the tumor bed with troglitazone and a chemotherapeutic drug.

Other methods of the present invention for treating a subject having a tumor comprise the steps of surgically revealing the tumor and contacting the tumor with troglitazone and a chemotherapeutic drug. Alternatively, a method for treating a subject having a tumor may comprise the step of perfusing the tumor, over an extended period of time, with troglitazone and a chemotherapeutic drug.

The use of the word “a” or “an” when used in conjunction with the term “comprising” in the claims and/or the specification may mean “one,” but it is also consistent with the meaning of “one or more,” “at least one,” and “one or more than one.”

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1A. DNA concentration of Saos-2 cells 3 days after a single treatment of troglitazone. FIG. 1A shows DNA content per well following a single treatment with troglitazone. * Denotes different from control (P<0.05).

FIG. 1B. LDH activity in Saos-2 Cells treated with troglitazone for 3 days. FIG. 1B shows lactate dehydrogenase (LDH) levels as measured in the media after 3 days of treatment.

FIG. 2. CAT activity in Saos-2 cells transfected with AOX-PPRE-tk-CAT construct. FIG. 2 shows that troglitazone treatment increase CAT activity 4 fold in the absence of transfected PPAR-γ (lane 2 vs lane 1) as well as the presence of transfected PPARγ (lanes 3 and 4). This demonstrates the existence of functional PPAR-γ in Saos-2 cells. * Denotes P<0.05 compared to appropriate control.

FIG. 3. ³H-Thymidine incorporation into Saos-2 cells. FIG. 3 shows that a dose of 5 μg/ml troglitazone caused a 16% reduction of ³H-uridine uptake while a 10 μg/ml dose caused a 43% reduction. * Denotes statistically different from control (P<0.05).

FIG. 4. mRNA levels in Saos-2 cells. FIG. 4 shows the effect upon mRNA levels for cell cycle inhibitor genes in response to a single treatment of 1 μM troglitazone for 24 hr. This revealed a 2.6 fold increase in p21 mRNA levels. Levels were normalized to GAPDH. Another control used is L32 which is seen to change very little. p18 is shown not change, as well.

FIG. 5. DNA content of Saos-2 cells 3 days after a single treatment of troglitazone and/or 5-fluorouracil. FIG. 5 shows the effects of toxic chemotherapeutic agent, 5-fluorouracil (5-FU), revealing that combinatorial therapy with 5 μM troglitazone and 1 μM 5-FU was equivalent to 100 μM dose of 5-FU alone. Combinatorial therapy reduces the dose of 5-FU needed by a factor of 100. * Denotes different from control (P<0.05); +Denotes different from corresponding 5-fluorouracil treatment (P<0.05).

FIG. 6. DNA content of Saos-2 cells. FIG. 6 shows that troglitazone demonstrates a similar lowering of the dose of doxorubicin needed to inhibit cell proliferation. * Denotes P<0.05 as compared to control; +denotes P<0.05 as troglitazone/doxorubicin compared to equivalent doxorubicin dose of doxorubicin. DNA was isolated from cells 4 days after a single treatment with the indicated drug.

FIG. 7. DNA content of Saos-2 cells. FIG. 7 shows that troglitazone may be acting or acting additionally through its vitamin E moiety in addition to its ability to bind to PPAR-Y.

FIG. 8. DNA content of Saos-2 cells. FIG. 8 shows the results of combinatorial therapy with 5-flourouracil (5-FU) and/or troglitazone, BRL49653, or pioglitazone. P<0.05 as compared to match contol with/without 5-fluorouracil (5-FU).

FIG. 9. DNA content of Saos-2 cells. FIG. 9 shows that the thiazolidinediones had no additional inhibitory effect on cell proliferation when administered with methotrexate but did with doxorubicin. * Denotes P<0.05 as compared to its doxorubicin control.

FIG. 10. DNA content of UMCR 3 cells following a single treatment of troglitazone for 3 days. Cells were plated at approximately 50,000 cells/well. The following day, media was changed and renewed including the appropriate dose of troglitazone. Three days following treatment, DNA content was determined. FIG. 10 shows a dose response inhibition of cell proliferation of human renal cell carcinoma (UMCR 3) from a single treatment with troglitazone.

FIG. 11. DNA content of UMCR 3 cells after treatment with 10 μM troglitazone and/or 5-fluorouracil (5-FU). Cells were plated at approximately 50,000 cells/well. The following day, media was changed and renewed including the appropriate compounds. Three days following treatment, DNA content was determined. * indicates significant difference (P<0.05) from groups treated with troglitazone or 5-fluorouracil alone. FIG. 11 shows that combinatorial therapy with troglitazone and 5-FU is more effective in inhibiting cell proliferation than monotherapy with either agent alone.

FIG. 12. DNA content of UMCR 3 cells after treatment with 10 μM troglitazone and/or doxorubicin. Cells were plated at approximately 50,000 cells/well. The following day, media was changed and renewed including the appropriate compounds. Three days following treatment, DNA content was determined. FIG. 12 demonstrates that combinatorial treatment of troglitazone and doxorubicin significantly inhibits cell proliferation greater than that with either agent alone.

FIG. 13. DNA content of UMCR 3 cells after treatment with different compounds. Cells were plated at approximately 50,000 cells/well. The following day, media was changed and renewed including the appropriate compounds. Three days following treatment, DNA content was determined. * indicates statistical difference (P<0.05) from control. FIG. 13 shows that single treatments of thiazolidinediones (2, 3, 4) inhibit cell proliferation in human renal cell carcinoma (UMCR 3) cells. Troglitazone is the most effective. Retinoic acids (5, 6) are much less effective than thiazolidinediones.

FIG. 14. DNA content of UMCR 6 cells after treatment with different compounds. Cells were plated at approximately 50,000 cells/well. The following day, media was changed and renewed including the appropriate compounds. Three days following treatment, DNA content was determined. * indicates statistical difference (P<0.05) from control. FIG. 14 shows that the thiazolidinediones (2, 3 4) inhibit cell proliferation and the retinoic acids (5, 6) have little effect.

FIG. 15. DNA content of UMCR 7 cells after treatment with different compounds. Cells were plated at approximately 50,000 cells/well. The following day, media was changed and renewed including the appropriate compounds. Three days following treatment, DNA content was determined. * indicates statistical difference (P<0.05) from control. FIG. 15 shows that the retinoic acid and thiazolidinediones, except troglitazone, have little inhibitory activity on cell proliferation.

FIG. 16. Demonstration of functional PPARy in UMCR cells. 10 μM Troglitazone (24-hour treatment) induces luciferase expression from a PPRE/luciferase plasmid (10 μg) transiently transfected into UMCR 3, 6, and 7 cells. FIG. 16 shows that UMCR 3 and 6 have functional PPARγ, but UMCR 7 cells do not.

FIG. 17. DNA content of human ovarian tumor cells expressed as percent of control with the following treatments: A. 10 μM pioglitazone, B. 10 μM rosiglitazone, and C. 10 μM troglitazone. Cells were plated at 50,000/cells/12 well plate for 24 h followed by a single treatment with the appropriate compound or placebo for control (0.1% DMSO). DNA content for each well was determined 3 days after treatment. The cell lines used are as follows: 1. CaOV3 2.222 3. PA-1 4.2774 5. OV CAR3 and 6. SK OV3 cells. FIG. 17 shows that different thiazolidinediones are more effective in different ovarian tumor cell types. Rosiglitazone and pioglitazone are most effective at inhibiting cell proliferation in CaOV3, PA-1, and OV CAR3 cells.

FIG. 18. DNA content of human ovarian 2774 cells treated with combinatorial therapy. A. taxol and troglitazone. B. taxol and cisplatin. Cells were plated at 50,000 cells/well for 24 hours in 5% FBS/DMEM. After 24 hours, media was removed and new media added with compounds added as indicated in the figures. FIG. 18 shows that combinatorial therapy is more effective than monotherapy in inhibiting cell proliferation.

### DESCRIPTION OF ILLUSTRATIVE EMBODIMENTS

An estimated number of 1,228,600 new cases of invasive cancer are expected in the United States in 1998. As life expectancy improves, and infant mortality and deaths from infectious diseases decline, the importance of therapies for treating patients having cancer increases.

One purpose of the present invention is to identify and/or develop nontoxic or low toxic chemotherapeutic regimens to treat cancer. The cells used are derived from an osteosarcoma from an eleven year girl who died after aggressive chemotherapeutic treatment with adriamycin, vincristine, cytoxan, and aramycin-C.

Troglitazone (prototypical thiazolidinedione) is used clinically to treat Type 2 diabetics and has been shown to have little toxic activity except in rare instances of idiosynchratic hepatic toxicity. Therefore, this invention demonstrates that troglitazone alone is not toxic and inhibits cell proliferation in Saos-2 cells by inhibiting cell cycle progression. Another important discovery described herein is that combinatorial therapy using troglitazone requires 100-fold less toxic chemotherapeutic agent 5-fluorouracil (5-FU) for equivalent inhibition of cell proliferation (as compared to 5-FU treatment alone). 5-FU is toxic to cells because it is a DNA base analog that inhibits DNA synthesis. Thus, its mechanism of action differs from that of troglitazone. This latter discovery supports a powerful technique allowing the use of lower concentrations of toxic chemotherapeutic agents while providing successful therapeutic results in causing tumor regression and curing cancer.

According to the invention, it is possible to use troglitazone or other thiazolidinedione compounds to inhibit tumor cell growth in osteosarcoma or other cancer cells. It is also possible to use the troglitazone or other thiazolidinedione compounds in combination with chemotherapeutic agents for the purpose of using lower doses of the toxic agent to effectively cause tumor regression. Thiazolidinediones other than troglitazone usable in the practice of the present invention are seen in U.S. Pat. No. 5,478,852, issued Dec. 26, 1995 and assigned to Sankyo Company, Limited, Tokyo, Japan.

The inventors have elucitated that troglitazone alone will inhibit cell proliferation of human osteosarcoma cells. One mechanism of action of troglitazone is its action through binding to the peroxisome proliferator activated receptor γ (PPAR-γ). PPAR-γ is a transcriptional factor that can cause terminal differentiation of adipocytes. PPAR-γ expression has not been previously demonstrated in osteosarcomas. It was also not known, prior to the inventor's work, that troglitazone could lower the IC₅₀ of toxic chemotherapeutic compounds when administered in combination with these toxic agents.

The inventive steps described herein include demonstrating that troglitazone inhibits cell proliferation in human osteosarcoma cells, the presence of functional PPAR-γ in such cells, and the demonstration of the ability of troglitazone to lower the IC₅₀ of other chemotherapeutic agents when administered in combination therewith as regarding cell proliferation. This concept can be extended to inhibiting cell growth or proliferation of cancer cells generally, including renal carcinoma cells and ovarian cancer cells. Moreover, lowering of the IC₅₀ of other chemotherapeutic agents has benefits in reducing the incidence of multidrug resistance (MDR) that can be observed as a result of chemotherapeutic treatment regimens.

I. Troglitazone and Other Thiazolidinediones

The methods of the present invention are directed at methods of inhibiting a pre-cancer or cancer cell and generally treating a subject with pre-cancer or cancer through the use of troglitazone or other thiazolidinediones.

Troglitazone (±5-[[4-[(3,4-dihydro-6-hydroxy-2, 5, 7, 8-tetramethyl-2H-1-benzopyran-2-yl)methoxy]phenyl]methyl]-2,4-thiazolidinedione) has been used in the management of Type 2 diabetes as an antihyperglycemic agent. Insulin sensitivity is enhanced by it in muscle and adipose tissue, and troglitazone impedes hepatic gluconeogenesis. It has been used alone or in combination with a sulfonylurea to control adult-onset diabetes. It has a molecular weight of approximately 441 daltons and its formula is C₂₄H₂₇NO₈S.

BRL49563 (rosiglitazone) and pioglitazone are second generation thiazolidinediones used in the treatment of type 2 diabetes (BRL49653 or avandia, Smith-Kline recently introduced on market). They were designed based on the structure of troglitazone and differ in that these two compounds do not contain the vitamin E moiety that is contained in troglitazone. BRL49653 and pioglitazone. These two compounds have a 100 and 6-fold higher affinity for the transcriptional factor PPARγ. Thus, in type 2 diabetes, 2-4 mg/day of BRL49563 (avandia, rosiglitazone) are used in patients as compared to 200-400 mg/day of troglitazone to lower blood glucose levels. It is important to note that thiazolidinediones do not cause hypoglycemia. These agents act to sensitize insulin's ability to cause blood glucose to be taken up into target cells e.g. adipocytes and muscle.

H. Methods for Assaying Troglitazone Activity

Inhibition of growth of cancer cells can measured using a Hoechst dye reagent, such as H33258, which is a well known DNA intercalating agent used to measure DNA levels. DNA content is directly proportional to cell number. A significant inhibition in growth is represented by decreases of at least about 30%40% as compared to uninhibited, and most preferably, of at least about 50%, with more significant decreases also being possible. Growth assays as measured by a Hoechst reagent are well known in the art. Assays may be conducted as described by Mosmann et al., 1983; Rubinstein et al., 1990 (both incorporated herein by reference). Therefore, if a candidate substance exhibited inhibition in this type of study, it would likely be a suitable compound for use in the present invention.

Quantitative in vitro testing is not a requirement of the invention as it is generally envisioned that the second agents will often be selected on the basis of their known properties or by structural and/or functional comparison to those agents disclosed herein. Therefore, the effective amounts will often be those amounts proposed to be safe for administration to animals in another context, for example, as disclosed herein. As the invention arises in part from the inventors' discovery of certain metabolic and physiological events, and the inventors' surprising combination of elements, there is considerable information available on the use and doses of second agents alone, which information may now be employed with the present invention.

III. Methods for Treating Cancer Using Troglitazone and Other Thiazolidinediones

A. Treatment of Cancer or Precancer

The present invention involves the treatment of cancer and precancer/preneoplastic conditions. The types of conditions that may be treated, according to the present invention, are limited only by the involvement of troglitazone. By involvement, it is meant that troglitazone inhibits the growth of a cancer cell or a tumor. The term “cancer cell” is used to indicate a cell whose growth is uncontrolled. In addition to cancers where a tumor is not formed, it is contemplated that a wide variety of tumors, including solid tumors, may be treated using troglitazone therapy, including cancers of the brain (glioblastoma, astrocytoma, oligodendroglioma, ependymomas), lung, liver, spleen, kidney, lymph node, pancreas, small intestine, colon, stomach, breast, endometrium, prostate, testicle, ovary, skin, head and neck, esophagus, bone marrow, blood and other tissue. Moreover, the treatment of pre-neoplastic conditions is also included since precancerous lesions can lead to the development of cancer. These pre-neoplastic conditions include, for example, oral hairy leukoplasia, bronchial dysplasia, carcinomas in situ, and intraepithelial hyperplasia.

In many contexts, it is not necessary that the cell be killed or induced to undergo normal cell death or “apoptosis.” Rather, to accomplish a meaningful treatment involving troglitazone, all that is required is that the growth of cancer cells or tumor growth be slowed to some degree. It may be that the cell's growth is completely blocked, however, or that some tumor regression is achieved. Clinical terminology such as “remission” and “reduction of tumor” burden also are contemplated given their normal usage.

The term “therapeutic benefit” used throughout this application refers to anything that promotes or enhances the well-being of the subject with respect to the medical treatment of his condition, which includes treatment of pre-cancer and cancer. A list of nonexhaustive examples of a “therapeutic benefit” includes extension of the subject's life by any period of time, decrease or delay in the neoplastic development of the disease, decrease in growth or proliferation of cancer cells, reduction in tumor growth, delay or prevention of metastases, reduction in cancer cell or tumor cell proliferation rate, and a decrease in pain to the subject that can be attributed to the subject's condition.

B. Chemotherapeutic Agents

A wide variety of chemotherapeutic agents may be used in combination with the use of troglitazone in the present invention. The term “chemotherapy” refers to the use of drugs to treat cancer. A “chemotherapeutic agent” is used to connote a compound or composition that is administered in the treatment of cancer. These agents or drugs are categorized by their mode of activity within a cell, for example, whether and at what stage they affect the cell cycle. Alternatively, an agent may be characterized based on its ability to directly cross-link DNA, to intercalate into DNA, or to induce chromosomal and mitotic aberrations by affecting nucleic acid synthesis. Most chemotherapeutic agents fall into the following categories: alkylating agents, antimetabolites, antitumor antibiotics, corticosteroid hormones, mitotic inhibitors, and nitrosoureas. It is contemplated that troglitazone can be used in combination with one or more of these agents according to the present invention.

1. Alkylating Agents

Alkylating agents are drugs that directly interact with genomic DNA to prevent the cancer cell from proliferating. This category of chemotherapeutic drugs represents agents that affect all phases of the cell cycle, that is, they are not phase-specific. Alkylating agents can be implemented to treat chronic leukemia, non-Hodgkin's lymphoma, Hodgkin's disease, multiple myeloma, and particular cancers of the breast, lung, and ovary. They include: busulfan, chlorambucil, cisplatin, cyclophosphamide (cytoxan), dacarbazine, ifosfamide, mechlorethamine (mustargen), and melphalan. Troglitazaone can be used to treat cancer in combination with any one or more of these alkylating agents, some of which are discussed below.

a. Busulfan

Busulfan (also known as myleran) is a bifunctional alkylating agent. Busulfan is known chemically as 1,4-butanediol dimethanesulfonate.

Busulfan is not a structural analog of the nitrogen mustards. Busulfan is available in tablet form for oral administration. Each scored tablet contains 2 mg busulfan and the inactive ingredients magnesium stearate and sodium chloride.

Busulfan is indicated for the palliative treatment of chronic myelogenous (myeloid, myelocytic, granulocytic) leukemia. Although not curative, busulfan reduces the total granulocyte mass, relieves symptoms of the disease, and improves the clinical state of the patient. Approximately 90% of adults with previously untreated chronic myelogenous leukemia will obtain hematologic remission with regression or stabilization of organomegaly following the use of busulfan. It has been shown to be superior to splenic irradiation with respect to survival times and maintenance of hemoglobin levels, and to be equivalent to irradiation at controlling splenomegaly.

b. Chlorambucil

Chlorambucil (also known as leukeran) is a bifunctional alkylating agent of the nitrogen mustard type that has been found active against selected human neoplastic diseases. Chlorambucil is known chemically as 4-[bis(2-chlorethyl)amino] benzenebutanoic acid.

Chlorambucil is available in tablet form for oral administration. It is rapidly and completely absorbed from the gastrointestinal tract. After single oral doses of 0.6-1.2 mg/kg, peak plasma chlorambucil levels are reached within one hour and the terminal half-life of the parent drug is estimated at 1.5 hours. 0.1 to 0.2 mg/kg/day or 3 to 6 mg/m²/day or alternatively 0.4 mg/kg may be used for antineoplastic treatment. Treatment regimes are well know to those of skill in the art and can be found in the “Physicians Desk Reference” and in “Remington's Pharmaceutical Sciences” referenced herein.

Chlorambucil is indicated in the treatment of chronic lymphatic (lymphocytic) leukemia, malignant lymphomas including lymphosarcoma, giant follicular lymphoma and Hodgkin's disease. It is not curative in any of these disorders but may produce clinically useful palliation. Thus, it can be used in combination with troglitazone in the treatment of cancer.

C. Cisplatin

Cisplatin has been widely used to treat cancers such as metastatic testicular or ovarian carcinoma, advanced bladder cancer, head or neck cancer, cervical cancer, lung cancer or other tumors. Cisplatin can be used alone or in combination with other agents, with efficacious doses used in clinical applications of 15-20 mg/m² for 5 days every three weeks for a total of three courses. Exemplary doses may be 0.50 mg/m², 1.0 mg/m², 1.50 mg/m², 1.75 mg/m², 2.0 mg/m², 3.0 mg/m², 4.0 mg/m², 5.0 mg/m², 110 mg/m². Of course, all of these dosages are exemplary, and any dosage in-between these points is also expected to be of use in the invention.

Cisplatin is not absorbed orally and must therefore be delivered via injection intravenously, subcutaneously, intratumorally or intraperitoneally.

d. Cyclophosphamide

Cyclophosphamide is 2H-1,3,2-Oxazaphosphorin-2-amine, N,N-bis(2-chloroethyl)tetrahydro-, 2-oxide, monohydrate; termed Cytoxan available from Mead Johnson; and Neosar available from Adria. Cyclophosphamide is prepared by condensing 3-amino-1-propanol with NN-bis(2-chlorethyl) phosphoramidic dichloride [(ClCH₂CH₂)₂N—POCl₂] in dioxane solution under the catalytic influence of triethylamine. The condensation is double, involving both the hydroxyl and the amino groups, thus effecting the cyclization.

Unlike other β-chloroethylamino alkylators, it does not cyclize readily to the active ethyleneimonium form until activated by hepatic enzymes. Thus, the substance is stable in the gastrointestinal tract, tolerated well and effective by the oral and parental routes and does not cause local vesication, necrosis, phlebitis or even pain.

Suitable doses for adults include, orally, 1 to 5 mg/kg/day (usually in combination), depending upon gastrointestinal tolerance; or 1 to 2 mg/kg/day; intravenously, initially 40 to 50 mg/kg in divided doses over a period of 2 to 5 days or 10 to 15 mg/kg every 7 to 10 days or 3 to 5 mg/kg twice a week or 1.5 to 3 mg/kg/day. A dose 250 mg/kg/day may be administered as an antineoplastic. Because of gastrointestinal adverse effects, the intravenous route is preferred for loading. During maintenance, a leukocyte count of 3000 to 4000/mm³ usually is desired. The drug also sometimes is administered intramuscularly, by infiltration or into body cavities. It is available in dosage forms for injection of 100, 200 and 500 mg, and tablets of 25 and 50 mg the skilled artisan is referred to “Remington's Pharmaceutical Sciences” 15th Edition, chapter 61, incorporate herein as a reference, for details on doses for administration.

e. Melphalan

Melphalan, also known as alkeran, L-phenylalanine mustard, phenylalanine mustard, L-PAM, or L-sarcolysin, is a phenylalanine derivative of nitrogen mustard. Melphalan is a bifunctional alkylating agent which is active against selective human neoplastic diseases. It is known chemically as 4-[bis(2-chloroethyl)amino]-L-phenylalanine.

Melphalan is the active L-isomer of the compound and was first synthesized in 1953 by Bergel and Stock; the D-isomer, known as medphalan, is less active against certain animal tumors, and the dose needed to produce effects on chromosomes is larger than that required with the L-isomer. The racemic (DL-) form is known as merphalan or sarcolysin. Melphalan is insoluble in water and has a pKa₁ of ˜2.1. Melphalan is available in tablet form for oral administration and has been used to treat multiple myeloma.

Available evidence suggests that about one third to one half of the patients with multiple myeloma show a favorable response to oral administration of the drug.

Melphalan has been used in the treatment of epithelial ovarian carcinoma. One commonly employed regimen for the treatment of ovarian carcinoma has been to administer melphalan at a dose of 0.2 mg/kg daily for five days as a single course. Courses are repeated every four to five weeks depending upon hematologic tolerance (Smith and Rutledge, 1975; Young et al., 1978). Alternatively the dose of melphalan used could be as low as 0.05 mg/kg/day or as high as 3 mg/kg/day or any dose in between these doses or above these doses. Some variation in dosage will necessarily occur depending on the condition of the subject being treated. The person responsible for administration will, in any event, determine the appropriate dose for the individual subject

2. Antimetabolites

Antimetabolites disrupt DNA and RNA synthesis. Unlike alkylating agents, they specifically influence the cell cycle during S phase. They have used to combat chronic leukemias in addition to tumors of breast, ovary and the gastrointestinal tract. Antimetabolites include 5-fluorouracil (5-FU), cytarabine (Ara-C), fludarabine, gemcitabine, and methotrexate.

a. 5-Fluorouracil

5-Fluorouracil (5-FU) has the chemical name of 5-fluoro-2,4(1H,3H)-pyrimidinedione. Its mechanism of action is thought to be by blocking the methylation reaction of deoxyuridylic acid to thymidylic acid. Thus, 5-FU interferes with the syntheisis of deoxyribonucleic acid (DNA) and to a lesser extent inhibits the formation of ribonucleic acid (RNA). Since DNA and RNA are essential for cell division and proliferation, it is thought that the effect of 5-FU is to create a thymidine deficiency leading to cell death. Thus, the effect of 5-FU is found in cells that rapidly divide, a characteristic of metastatic cancers.

3. Antitumor Antibiotics

Antitumor antibiotics have both antimicrobial and cytotoxic activity. These drugs also interfere with DNA by chemically inhibiting enzymes and mitosis or altering cellular membranes. These agents are not phase specific so they work in all phases of the cell cycle. Thus, they are widely used for a variety of cancers. Examples of antitumor antibiotics include bleomycin, dactinomycin, daunorubicin, doxorubicin (Adriamycin), and idarubicin, some of which are discussed in more detail below. Widely used in clinical setting for the treatment of neoplasms these compounds are administered through bolus injections intravenously at doses ranging from 25-75 mg/m² at 21 day intervals for adriamycin, to 35-100 mg/m² for etoposide intravenously or orally.

a. Doxorubicin

Doxorubicin hydrochloride, 5,12-Naphthacenedione, (8s-cis)-10-[(3-amino-2,3,6-trideoxy-a-L-lyxo-hexopyranosyl)oxy]-7,8,9,10-tetrahydro-6,8,11-trihydroxy-8-(hydroxyacetyl)-1-methoxy-hydrochloride (hydroxydaunorubicin hydrochloride, Adriamycin) is used in a wide antineoplastic spectrum. It binds to DNA and inhibits nucleic acid synthesis, inhibits mitosis and promotes chromosomal aberrations.

Administered alone, it is the drug of first choice for the treatment of thyroid adenoma and primary hepatocellular carcinoma. It is a component of 31 first-choice combinations for the treatment of ovarian, endometrial and breast tumors, bronchogenic oat-cell carcinoma, non-small cell lung carcinoma, gastric adenocarcinoma, retinoblastoma, neuroblastoma, mycosis fungoides, pancreatic carcinoma, prostatic carcinoma, bladder carcinoma, myeloma, diffuse histiocytic lymphoma, Wilms' tumor, Hodgkin's disease, adrenal tumors, osteogenic sarcoma soft tissue sarcoma, Ewing's sarcoma, rhabdomyosarcoma and acute lymphocytic leukemia. It is an alternative drug for the treatment of islet cell, cervical, testicular and adrenocortical cancers. It is also an immunosuppressant.

Doxorubicin is absorbed poorly and must be administered intravenously. The pharmacokinetics are multicompartmental. Distribution phases have half-lives of 12 minutes and 3.3 hr. The elimination half-life is about 30 hr. Forty to 50% is secreted into the bile. Most of the remainder is metabolized in the liver, partly to an active metabolite (doxorubicinol), but a few percent is excreted into the urine. In the presence of liver impairment, the dose should be reduced.

Appropriate doses are, intravenous, adult, 60 to 75 mg/m² at 21-day intervals or 25 to 30 mg/m² on each of 2 or 3 successive days repeated at 3- or 4-wk intervals or 20 mg/m² once a week. The lowest dose should be used in elderly patients, when there is prior bone-marrow depression caused by prior chemotherapy or neoplastic marrow invasion, or when the drug is combined with other myelopoietic suppressant drugs. The dose should be reduced by 50% if the serum bilirubin lies between 1.2 and 3 mg/dL and by 75% if above 3 mg/dL. The lifetime total dose should not exceed 550 mg/m² in patients with normal heart function and 400 mg/m² in persons having received mediastinal irradiation. Alternatively, 30 mg/m² on each of 3 consecutive days, repeated every 4 wk. Exemplary doses may be 10 mg/m², 20 mg/m², 30 mg/m², 50 mg/m², 100 mg/m², 150 mg/m2, 175 mg/m², 200 mg/m², 225 mg/m², 250 mg/m², 275 mg/m², 300 mg/m², 350 mg/m2,400 mg/m², 425 mg/m², 450 mg/m², 475 mg/m², 500 mg/m². Of course, all of these dosages are exemplary, and any dosage in-between these points is also expected to be of use in the invention.

In the present invention the inventors have employed troglitazone as an exemplary chemotherapeutic agent to synergistically enhance the antineoplastic effects of the doxorubicin in the treatment of cancers. Those of skill in the art will be able to use the invention as exemplified potentiate the effects of doxorubicin in a range of different pre-cancer and cancers.

b. Daunorubicin

Daunorubicin hydrochloride, 5,12-Naphthacenedione, (8S-cis)-8-acetyl-10-[(3-amino-2,3,6-trideoxy-a-L-lyxo-hexanopyranosyl)oxy]-7,8,9,10-tetrahydro-6,8,11-trihydroxy-10-methoxy-, hydrochloride; also termed cerubidine and available from Wyeth. Daunorubicin intercalates into DNA, blocks DAN-directed RNA polymerase and inhibits DNA synthesis. It can prevent cell division in doses that do not interfere with nucleic acid synthesis.

In combination with other drugs it is included in the first-choice chemotherapy of acute myelocytic leukemia in adults (for induction of remission), acute lymphocytic leukemia and the acute phase of chronic myelocytic leukemia. Oral absorption is poor, and it must be given intravenously. The half-life of distribution is 45 minutes and of elimination, about 19 hr. The half-life of its active metabolite, daunorubicinol, is about 27 hr. Daunorubicin is metabolized mostly in the liver and also secreted into the bile (ca 40%). Dosage must be reduced in liver or renal insufficiencies.

Suitable doses are (base equivalent), intravenous adult, younger than 60 yr. 45 mg/m²/day (30 mg/m² for patients older than 60 yr.) for 1, 2 or 3 days every 3 or 4 wk or 0.8 mg/kg/day for 3 to 6 days every 3 or 4 wk; no more than 550 mg/m² should be given in a lifetime, except only 450 mg/m² if there has been chest irradiation; children, 25 mg/m² once a week unless the age is less than 2 yr. or the body surface less than 0.5 m, in which case the weight-based adult schedule is used. It is available in injectable dosage forms (base equivalent) 20 mg (as the base equivalent to 21.4 mg of the hydrochloride). Exemplary doses may be 10 mg/m², 20 mg/m², 30 mglm², 50 mg/m², 100 mg/m², 150 mg/m², 175 mg/m², 200 mg/m², 225 mg/m², 250 mg/m², 275 mg/m², 300 mg/m², 350 mg/m², 400 mg/m², 425 mg/m², 450 mg/m², 475 mg/m², 500 mg/m². Of course, all of these dosages are exemplary, and any dosage in-between these points is also expected to be of use in the invention.

C. Mitomycin

Mitomycin (also known as mutamycin and/or mitomycin-C) is an antibiotic isolated from the broth of Streptomyces caespitosus which has been shown to have antitumor activity. The compound is heat stable, has a high melting point, and is freely soluble in organic solvents.

Mitomycin selectively inhibits the synthesis of deoxyribonucleic acid (DNA). The guanine and cytosine content correlates with the degree of mitomycin-induced cross-linking. At high concentrations of the drug, cellular RNA and protein synthesis are also suppressed.

In humans, mitomycin is rapidly cleared from the serum after intravenous administration. Time required to reduce the serum concentration by 50% after a 30 mg. bolus injection is 17 minutes. After injection of 30 mg., 20 mg., or 10 mg. I.V., the maximal serum concentrations were 2.4 mg./mL, 1.7 mg./mL, and 0.52 mg./mL, respectively. Clearance is effected primarily by metabolism in the liver, but metabolism occurs in other tissues as well. The rate of clearance is inversely proportional to the maximal serum concentration because, it is thought, of saturation of the degradative pathways. Approximately 10% of a dose of mitomycin is excreted unchanged in the urine. Since metabolic pathways are saturated at relatively low doses, the percent of a dose excreted in urine increases with increasing dose. In children, excretion of intravenously administered mitomycin is similar.

d. Actinomycin D

Actinomycin D (Dactinomycin) [50-76-0]; C₆₂H₈₆N₁₂O₁₆ (1255.43) is an antineoplastic drug that inhibits DNA-dependent RNA polymerase. It is a component of first-choice combinations for treatment of choriocarcinoma, embryonal rhabdomyosarcoma, testicular tumor and Wilms' tumor. Tumors that fail to respond to systemic treatment sometimes respond to local perfusion. Dactinomycin potentiates radiotherapy. It is a secondary (efferent) immunosuppressive.

Actinomycin D is used in combination with primary surgery, radiotherapy, and other drugs, particularly vincristine and cyclophosphamide. Antineoplastic activity has also been noted in Ewing's tumor, Kaposi's sarcoma, and soft-tissue sarcomas. Dactinomycin can be effective in women with advanced cases of choriocarcinoma. It also produces consistent responses in combination with chlorambucil and methotrexate in patients with metastatic testicular carcinomas. A response may sometimes be observed in patients with Hodgkin's disease and non-Hodgkin's lymphomas. Dactinomycin has also been used to inhibit immunological responses, particularly the rejection of renal transplants.

Half of the dose is excreted intact into the bile and 10% into the urine; the half-life is about 36 hr. The drug does not pass the blood-brain barrier. Actinomycin D is supplied as a lyophilized powder (0/5 mg in each vial). The usual daily dose is 10 to 15 mg/kg; this is given intravenously for 5 days; if no manifestations of toxicity are encountered, additional courses may be given at intervals of 3 to 4 weeks. Daily injections of 100 to 400 mg have been given to children for 10 to 14 days; in other regimens, 3 to 6 mg/kg, for a total of 125 mg/kg, and weekly maintenance doses of 7.5 mg/kg have been used. Although it is safer to administer the drug into the tubing of an intravenous infusion, direct intravenous injections have been given, with the precaution of discarding the needle used to withdraw the drug from the vial in order to avoid subcutaneous reaction. Exemplary doses may be 100 mg/m², 150 mg/m², 175 mg/m², 200 mg/m², 225 mg/m², 250 mg/m², 275 mg/m², 300 mg/m², 350 mg/m², 400 mg/m², 425 mg/m², 450 mg/m², 475 mg/m², 500 mg/m². Of course, all of these dosages are exemplary, and any dosage in-between these points is also expected to be of use in the invention.

e. Bleomycin

Bleomycin is a mixture of cytotoxic glycopeptide antibiotics isolated from a strain of Streptomyces verticillus. Although the exact mechanism of action of bleomycin is unknown, available evidence would seem to indicate that the main mode of action is the inhibition of DNA synthesis with some evidence of lesser inhibition of RNA and protein synthesis.

In mice, high concentrations of bleomycin are found in the skin, lungs, kidneys, peritoneum, and lymphatics. Tumor cells of the skin and lungs have been found to have high concentrations of bleomycin in contrast to the low concentrations found in hematopoietic tissue. The low concentrations of bleomycin found in bone marrow may be related to high levels of bleomycin degradative enzymes found in that tissue.

In patients with a creatinine clearance of >35 mL per minute, the serum or plasma terminal elimination half-life of bleomycin is approximately 115 minutes. In patients with a creatinine clearance of <35 mL per minute, the plasma or serum terminal elimination half-life increases exponentially as the creatinine clearance decreases. In humans, 60% to 70% of an administered dose is recovered in the urine as active bleomycin. Bleomycin may be given by the intramuscular, intravenous, or subcutaneous routes. It is freely soluble in water.

Bleomycin should be considered a palliative treatment. It has been shown to be useful in the management of the following neoplasms either as a single agent or in proven combinations with other approved chemotherapeutic agents in squamous cell carcinoma such as head and neck (including mouth, tongue, tonsil, nasopharynx, oropharynx, sinus, palate, lip, buccal mucosa, gingiva, epiglottis, larynx), skin, penis, cervix, and vulva. It has also been used in the treatment of lymphomas and testicular carcinoma.

Because of the possibility of an anaphylactoid reaction, lymphoma patients should be treated with two units or less for the first two doses. If no acute reaction occurs, then the regular dosage schedule may be followed.

Improvement of Hodgkin's Disease and testicular tumors is prompt and noted within 2 weeks. If no improvement is seen by this time, improvement is unlikely. Squamous cell cancers respond more slowly, sometimes requiring as long as 3 weeks before any improvement is noted.

4. Corticosteroid Hormones

Corticosteroid hormones are useful in treating some types of cancer (lymphoma, leukemias, and multiple myeloma). Though these hormones have been used in the treatment of many non-cancer conditions, they are considered chemotherapy drugs when they are implemented to kill or slow the growth of cancer cells. Like troglitazone, corticosteroid hormones can increase the effectiveness of other chemotherapy agents, and consequently, they are frequently used in combination treatments. Prednisone and dexamethasone are examples of corticosteroid hormones.

5. Mitotic Inhibitors

Mitotic inhibitors include plant alkaloids and other natural agents that can inhibit either protein synthesis required for cell division or mitosis. They operate during a specific phase during the cell cycle. Mitotic inhibitors comprise docetaxel, etoposide (VP16), paclitaxel, taxol, vinblastine, vincristine, and vinorelbine.

a. Etoposide (VP16)

VP16 is also known as etoposide and is used primarily for treatment of testicular tumors, in combination with bleomycin and cisplatin, and in combination with cisplatin for small-cell carcinoma of the lung. It is also active against non-Hodgkin's lymphomas, acute nonlymphocytic leukemia, carcinoma of the breast, and Kaposi's sarcoma associated with acquired immunodeficiency syndrome (AIDS).

VP16 is available as a solution (20 mg/ml) for intravenous administration and as 50-mg, liquid-filled capsules for oral use. For small-cell carcinoma of the lung, the intravenous dose (in combination therapy) is can be as much as 100 mg/m² or as little as 2 mg/m², routinely 35 mg/m², daily for 4 days, to 50 mg/m², daily for 5 days have also been used. When given orally, the dose should be doubled. Hence the doses for small cell lung carcinoma may be as high as 200-250 mg/m². The intravenous dose for testicular cancer (in combination therapy) is 50 to 100 mg/m² daily for 5 days, or 100 mg/m² on alternate days, for three doses. Cycles of therapy are usually repeated every 3 to 4 weeks. The drug should be administered slowly during a 30- to 60-minute infusion in order to avoid hypotension and bronchospasm, which are probably due to the solvents used in the formulation.

b. Taxol

Taxol is an experimental antimitotic agent, isolated from the bark of the ash tree, Taxus brevifolia. It binds to tubulin (at a site distinct from that used by the vinca alkaloids) and promotes the assembly of microtubules. Taxol is currently being evaluated clinically; it has activity against malignant melanoma and carcinoma of the ovary. Maximal doses are 30 mg/m² per day for 5 days or 210 to 250 mg/m² given once every 3 weeks. Of course, all of these dosages are exemplary, and any dosage in-between these points is also expected to be of use in the invention.

C. Vinblastine

Vinblastine is another example of a plant aklyloid that can be used in combination with troglitazone for the treatment of cancer and precancer. When cells are incubated with vinblastine, dissolution of the microtubules occurs.

Unpredictable absorption has been reported after oral administration of vinblastine or vincristine. At the usual clinical doses the peak concentration of each drug in plasma is approximately 0.4 mM. Vinblastine and vincristine bind to plasma proteins. They are extensively concentrated in platelets and to a lesser extent in leukocytes and erythrocytes.

After intravenous injection, vinblastine has a multiphasic pattern of clearance from the plasma; after distribution, drug disappears from plasma with half-lives of approximately 1 and 20 hours. Vinblastine is metabolized in the liver to biologically activate derivative desacetylvinblastine. Approximately 15% of an administered dose is detected intact in the urine, and about 10% is recovered in the feces after biliary excretion. Doses should be reduced in patients with hepatic dysfunction. At least a 50% reduction in dosage is indicated if the concentration of bilirubin in plasma is greater than 3 mg/dl (about 50 mM).

Vinblastine sulfate is available in preparations for injection. The drug is given intravenously; special precautions must be taken against subcutaneous extravasation, since this may cause painful irritation and ulceration. The drug should not be injected into an extremity with impaired circulation. After a single dose of 0.3 mg/kg of body weight, myelosuppression reaches its maximum in 7 to 10 days. If a moderate level of leukopenia (approximately 3000 cells/mm³) is not attained, the weekly dose may be increased gradually by increments of 0.05 mg/kg of body weight. In regimens designed to cure testicular cancer, vinblastine is used in doses of 0.3 mg/kg every 3 weeks irrespective of blood cell counts or toxicity.

The most important clinical use of vinblastine is with bleomycin and cisplatin in the curative therapy of metastatic testicular tumors. Beneficial responses have been reported in various lymphomas, particularly Hodgkin's disease, where significant improvement may be noted in 50 to 90% of cases. The effectiveness of vinblastine in a high proportion of lymphomas is not diminished when the disease is refractory to alkylating agents. It is also active in Kaposi's sarcoma, neuroblastoma, and Letterer-Siwe disease (histiocytosis X), as well as in carcinoma of the breast and choriocarcinoma in women.

Doses of vinblastine will be determined by the clinician according to the individual patients need. 0.1 to 0.3 mg/kg can be administered or 1.5 to 2 mg/m² can also be administered. Alternatively, 0.1 mg/m², 0.12 mg/m², 0.14 mg/m², 0.15 mg/m², 0.2 mg/m², 0.25 mg/m², 0.5 mg/m², 1.0 mg/m², 1.2 mg/m², 1.4 mg/m², 1.5 mg/m², 2.0 mg/m², 2.5 mg/m², 5.0 mg/m², 6 mg/m², 8 mg/m², 9 mg/m², 10 mg/m², 20 mg/m², can be given. Of course, all of these dosages are exemplary, and any dosage in-between these points is also expected to be of use in the invention.

d. Vincristine

Vincristine blocks mitosis and produces metaphase arrest. It seems likely that most of the biological activities of this drug can be explained by its ability to bind specifically to tubulin and to block the ability of protein to polymerize into microtubules. Through disruption of the microtubules of the mitotic apparatus, cell division is arrested in metaphase. The inability to segregate chromosomes correctly during mitosis presumably leads to cell death.

The relatively low toxicity of vincristine for normal marrow cells and epithelial cells make this agent unusual among anti-neoplastic drugs, and it is often included in combination with other myelosuppressive agents.

Unpredictable absorption has been reported after oral administration of vinblastine or vincristine. At the usual clinical doses the peak concentration of each drug in plasma is approximately 0.4 mM.

Vinblastine and vincristine bind to plasma proteins. They are extensively concentrated in platelets and to a lesser extent in leukocytes and erythrocytes.

Vincristine has a multiphasic pattern of clearance from the plasma; the terminal half-life is about 24 hours. The drug is metabolized in the liver, but no biologically active derivatives have been identified. Doses should be reduced in patients with hepatic dysfunction. At least a 50% reduction in dosage is indicated if the concentration of bilirubin in plasma is greater than 3 mg/dl (about 50 mM).

Vincristine sulfate is available as a solution (1 mg/ml) for intravenous injection. Vincristine used together with corticosteroids is presently the treatment of choice to induce remissions in childhood leukemia; the optimal dosages for these drugs appear to be vincristine, intravenously, 2 mg/m² of body-surface area, weekly, and prednisone, orally, 40 mg/m², daily. Adult patients with Hodgkin's disease or non-Hodgkin's lymphomas usually receive vincristine as a part of a complex protocol. When used in the MOPP regimen, the recommended dose of vincristine is 1.4 mg/m². High doses of vincristine seem to be tolerated better by children with leukemia than by adults, who may experience sever neurological toxicity. Administration of the drug more frequently than every 7 days or at higher doses seems to increase the toxic manifestations without proportional improvement in the response rate. Precautions should also be used to avoid extravasation during intravenous administration of vincristine. Vincristine (and vinblastine) can be infused into the arterial blood supply of tumors in doses several times larger than those that can be administered intravenously with comparable toxicity.

Vincristine has been effective in Hodgkin's disease and other lymphomas. Although it appears to be somewhat less beneficial than vinblastine when used alone in Hodgkin's disease, when used with mechlorethamine, prednisone, and procarbazine (the so-called MOPP regimen), it is the preferred treatment for the advanced stages (III and IV) of this disease. In non-Hodgkin's lymphomas, vincristine is an important agent, particularly when used with cyclophosphamide, bleomycin, doxorubicin, and prednisone. Vincristine is more useful than vinblastine in lymphocytic leukemia. Beneficial response have been reported in patients with a variety of other neoplasms, particularly Wilms' tumor, neuroblastoma, brain tumors, rhabdomyosarcoma, and carcinomas of the breast, bladder, and the male and female reproductive systems.

Doses of vincristine for use will be determined by the clinician according to the individual patients need. 0.01 to 0.03 mg/kg or 0.4 to 1.4 mg/m² can be administered or 1.5 to 2 mg/m2 can alos be administered. Alternatively 0.02 mg/m², 0.05 mg/m², 0.06 mg/m2, 0.07 mg/m², 0.08 mg/m², 0.1 mg/m², 0.12 mg/m², 0.14 mg/m², 0.15 mg/m², 0.2 mg/m², 0.25 mg/m² can be given as a constant intravenous infusion. Of course, all of these dosages are exemplary, and any dosage in-between these points is also expected to be of use in the invention.

6. Nitrosureas

Nitrosureas, like alkylating agents, inhibit DNA repair proteins. They are used to treat non-Hodgkin's lymphomas, multiple myeloma, malignant melanoma, in addition to brain tumors. Examples include carmustine and lomustine.

a. Carmustine

Carmustine (sterile carmustine) is one of the nitrosoureas used in the treatment of certain neoplastic diseases. It is 1,3bis (2-chloroethyl)-1-nitrosourea. It is lyophilized pale yellow flakes or congealed mass with a molecular weight of 214.06. It is highly soluble in alcohol and lipids, and poorly soluble in water. Carmustine is administered by intravenous infusion after reconstitution as recommended. The structural formula is:

Sterile carmustine is commonly available in 100 mg single dose vials of lyophilized material.

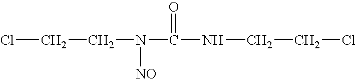

Although it is generally agreed that carmustine alkylates DNA and RNA, it is not cross resistant with other alkylators. As with other nitrosoureas, it may also inhibit several key enzymatic processes by carbamoylation of amino acids in proteins.

Carmustine is indicated as palliative therapy as a single agent or in established combination therapy with other approved chemotherapeutic agents in brain tumors such as glioblastoma, brainstem glioma, medullobladyoma, astrocytoma, ependymoma, and metastatic brain tumors. Also it has been used in combination with prednisone to treat multiple myeloma. Carmustine has proved useful, in the treatment of Hodgkin's Disease and in non-Hodgkin's lymphomas, as secondary therapy in combination with other approved drugs in patients who relapse while being treated with primary therapy, or who fail to respond to primary therapy.

The recommended dose of carmustine as a single agent in previously untreated patients is 150 to 200 mg/m² intravenously every 6 weeks. This may be given as a single dose or divided into daily injections such as 75 to 100 mg/m² on 2 successive days. When carmustine is used in combination with other myelosuppressive drugs or in patients in whom bone marrow reserve is depleted, the doses should be adjusted accordingly. Doses subsequent to the initial dose should be adjusted according to the hematologic response of the patient to the preceding dose. It is of course understood that other doses may be used in the present invention for example 10 mg/m², 20 mg/m², 30 mg/m² 40 mg/m² 50 mg/m² 60 mg/m² 70 mg² 80 mg/m² 90 mg/m² 100 mg/m². The skilled artisan is directed to, “Remington's Pharmaceutical Sciences” 15th Edition, chapter 61. Some variation in dosage will necessarily occur depending on the condition of the subject being treated. The person responsible for administration will, in any event, determine the appropriate dose for the individual subject.

b. Lomustine

Lomustine is one of the nitrosoureas used in the treatment of certain neoplastic diseases. It is 1-(2-chloro-ethyl)-3-cyclohexyl-1 nitrosourea. It is a yellow powder with the empirical formula of C₉H₁₆ClN₃O₂ and a molecular weight of 233.71. Lomustine is soluble in 10% ethanol (0.05 mg per mL) and in absolute alcohol (70 mg per mL). Lomustine is relatively insoluble in water (<0.05 mg per mL). It is relatively unionized at a physiological pH. Inactive ingredients in lomustine capsules are: magnesium stearate and mannitol.

Although it is generally agreed that lomustine alkylates DNA and RNA, it is not cross resistant with other alkylators. As with other nitrosoureas, it may also inhibit several key enzymatic processes by carbamoylation of amino acids in proteins.

Lomustine may be given orally. Following oral administration of radioactive lomustine at doses ranging from 30 mg/m² to 100 mg/m², about half of the radioactivity given was excreted in the form of degradation products within 24 hours. The serum half-life of the metabolites ranges from 16 hours to 2 days. Tissue levels are comparable to plasma levels at 15 minutes after intravenous administration.

Lomustine has been shown to be useful as a single agent in addition to other treatment modalities, or in established combination therapy with other approved chemotherapeutic agents in both primary and metastatic brain tumors, in patients who have already received appropriate surgical and/or radiotherapeutic procedures. It has also proved effective in secondary therapy against Hodgkin's Disease in combination with other approved drugs in patients who relapse while being treated with primary therapy, or who fail to respond to primary therapy.

The recommended dose of lomustine in adults and children as a single agent in previously untreated patients is 130 mg/m² as a single oral dose every 6 weeks. In individuals with compromised bone marrow function, the dose should be reduced to 100 mg/m² every 6 weeks. When lomustine is used in combination with other myelosuppressive drugs, the doses should be adjusted accordingly. It is understood that other doses may be used for example, 20 mg/m² 30 mg/m², 40 mg/m², 50 mg/m², 60 mg/m², 70 mg/m², 80 Mg/m², 90 mg/m², 100 mg/m², 120 mg/m² or any doses between these figures as determined by the clinician to be necessary for the individual being treated.

7. Miscellaneous Agents

Some chemotherapy agents do not qualify into the previous categories based on their activities. However, it is contemplated that they are included within the method of the present invention for use in combination therapies of cancer with troglitazone. They include amsacrine, L-asparaginase, tretinoin, and Tumor Necrosis Factor (TNF), some of which are discussed below.

a. Tumor Necrosis Factor

Tumor Necrosis Factor [TNF; Cachectin] is a glycoprotein that kills some kinds of cancer cells, activates cytokine production, activates macrophages and endothelial cells, promotes the production of collagen and collagenases, is an inflammatory mediator and also a mediator of septic shock, and promotes catabolism, fever and sleep. Some infectious agents cause tumor regression through the stimulation of TNF production. TNF can be quite toxic when used alone in effective doses, so that the optimal regimens probably will use it in lower doses in combination with other drugs. Its immunosuppressive actions are potentiated by gamma-interferon, so that the combination potentially is dangerous. A hybrid of TNF and interferon-α also has been found to possess anti-cancer activity.

In addition to combination treatment therapies comprising troglitazone or thiazolidinediones and another chemotherapeutic agent, it is also contemplated that the present invention includes the use of sex hormones according to the methods described herein in the treatment of cancer. While this method is not limited to the treatment of a specific cancer, this use of hormones in this combination therapy has benefits with respect to cancers of the breast, prostate, and endometrial (lining of the uterus). Examples of these hormones are estrogens, anti-estrogens, progesterones, and androgens.

C. Screening for Effectiveness of Cancer Treatment Using Animal Models

Animal models may be used as a screen for the cancer treatment effects of troglitazone and other thiazolidinedione compounds alone and in combination with other chemotherapeutic agents. Preferably, orthotopic animal models will be used so as to closely mimic the particular disease type being studied and to provide the most relevant results.

One type of orthotopic model involves the development of an animal model for the analysis of microscopic residual carcinomas and microscopic seeding of body cavities. “Carcinoma,” as used herein, may refer to a single cell or a multicellular tumor mass. In microscopic disease, the “tumor” will consist or one or a few carcinoma cells that cannot be observed with the naked eye. The animal model described herein is particularly advantageous mimicking (i) the post surgical environment of head and neck cancer patients, particularly in advanced stages of disease and (ii) the body cavity of an affected subject wherein microscopic carcinoma has been established. The model, similar to other animal models for cancer, derives from inoculation of tumor cells into an animal. A distinction, however, lies in the creation, subcutaneously, of a pouch that is a physiologic equivalent of a natural body cavity or a post-surgical cavity created by the excision of a tumor mass.

The instant invention preferably uses nude mice as the model organism. Virtually any animal may be employed, however, for use according to the present invention. Particularly preferred animals will be small mammals that are routinely used in laboratory protocols. Even more preferred animals are those of the rodent group, such as mice, rats, guinea pigs and hamsters. Rabbits also are a preferred species. The criteria for choosing an animal will be largely dependent upon the particular preference of an investigator.

The first step is to create a tissue flap in the experimental animal. The term “tissue flap” means any incision in the flesh of the animal that exposes the target tissue. It is generally preferred that an incision be made in the dorsal flank of an animal, as this represents a readily accessible site. However, it will be understood that an incision could well be made at other points on the animal, and the choice of tissue sites may be dependent upon various factors such as the particular type of therapeutics that are being investigated.

Once a target tissue site is exposed, carcinoma cells, either individually or in microscopic tumors, are contacted with the tissue site. The most convenient manner for seeding the cancer cells into the tissue site is to apply a suspension of tissue culture media containing the cells to the exposed tissue. Cancer cell application may be achieved simply using a sterile pipette or any other convenient applicator. Naturally, this procedure will be conducted under sterile conditions.

In a particular example, 2.5×10⁶ cells are inoculated into the exposed tissue flap of a nude mouse. Those of skill in the art will be able to readily determine, for a given purpose, what the appropriate number of cells will be. The number of cells will be dependent upon various factors, such as the size of the animal, the site of incision, the replicative capacity of the tumor cells themselves, the time intended for tumor growth, the potential anti-tumor therapeutic to be tested, and the like. Although establishing an optimal model system for any particular type of tumor may require a certain adjustment in the number of cells administered, this in no way represents an undue amount of experimentation. Those skilled in the area of animal testing will appreciate that such optimization is required.

This can be accomplished, for example, by conducting preliminary studies in which differing numbers of cells are delivered to the animal and the cell growth is monitored following resealing of the tissue flap. Naturally, administering larger numbers of cells will result in a larger population of microscopic residual tumor cells.

In the present study the flaps were effectively sealed using mattress sutures. However, it is envisioned that persons skilled in the art may use any of a variety of methods routinely used to seal the incision such as the use of adhesives, clamps, stitches, sutures, etc., depending on the particular use contemplated.

Other orthotopic animal models are well known in the art. The orthotopic lung cancer model, for example has been described in the literature. This protocol involves injection of tumor cells into the bronchus of a mouse wherein tumors will form in the bronchus and bronchioles, mimicking tumors commonly found in non-small cell lung cancer patients. The skilled artisan will readily be able to adapt or modify each particular model for his intended purpose without undue experimentation.

D. Combined Therapy with Immunotherapy, Traditional Chemotherapy, Radiotherapy or Other Anti-Cancer Agents

Tumor cell resistance to DNA damaging agents represents a major problem in clinical oncology. One goal of current cancer research is to find ways to improve the efficacy of chemo- and radiotherapy. One way is by combining therapies with agents, such as troglitazone, that increase the effectiveness of existing therapies and/or reduce their side effects. For example, the herpes simplex-thymidine kinase (HS-tk) gene, when delivered to brain tumors by a retroviral vector system, successfully induced susceptibility to the antiviral agent gancyclovir (Culver et al., 1992). In the context of the present invention, it is contemplated that troglitazone or other thiazolidinedione compound therapy could be used similarly in conjunction with anti-cancer agents, including chemo- or radiotherapeutic intervention. It also may prove effective to combine troglitozone with immunotherapy that targets cancer/tumor cells.

To kill cells, inhibit cell growth, inhibit metastasis, inhibit angiogenesis or otherwise reverse or reduce the malignant phenotype of cancer cells, using the methods and compositions of the present invention, one would generally contact a “target” cell with trolgitazone or another thiazolidinedione and at least one other agent. These compositions would be provided in a combined amount effective to kill or inhibit proliferation of the cell. This process may involve contacting the cells with troglitzone and the agent(s) or factor(s) at the same time. This may be achieved by contacting the cell with a single composition or pharmacological formulation that includes both agents, or by contacting the cell with two distinct compositions or formulations, at the same time, wherein one composition includes the expression construct and the other includes the agent.

Alternatively, the gene therapy treatment may precede or follow the other agent treatment by intervals ranging from minutes to weeks. In embodiments where the other agent and expression construct are applied separately to the cell, one would generally ensure that a significant period of time did not expire between the time of each delivery, such that the agent and expression construct would still be able to exert an advantageously combined effect on the cell. In such instances, it is contemplated that one would contact the cell with both modalities within about 12-24 hours of each other and, more preferably, within about 612 hours of each other, with a delay time of only about 12 hours being most preferred. In some situations, it may be desirable to extend the time period for treatment significantly, however, where several days (2, 3, 4, 5, 6 or 7) to several weeks (1, 2, 3, 4, 5, 6, 7 or 8) lapse between the respective administrations.

It also is conceivable that more than one administration of either troglitazone or the other agent will be desired. Various combinations may be employed, where troghtazone is “A” and the other agent is “B”, as exemplified below:

A/B/A B/A/B B/B/A A/A/B B/A/A A/B/B B/B/B/A B/B/A/B

A/A/B/B A/B/A/B A/B/B/A B/B/A/A B/A/B/A B/A/A/B B/B/B/A

A/A/A/B B/A/A/A A/B/A/A A/A/B/A A/B/B/B B/A/B/B B/B/A/B

Other combinations are contemplated. Again, to achieve cell killing, both agents are delivered to a cell in a combined amount effective to kill the cell.

Agents or factors suitable for use in a combined therapy are any chemical compound or treatment method with anticancer activity; therefore, the term “anticancer agent” that is used throughout this application refers to an agent with anticancer activity. These compounds or methods include alkylating agents, topoisomerase I inhibitors, topoisomerase II inhibitors, antitumor antibiotics, RNA/DNA antimetabolites, DNA antimetabolites, antimitotic agents, nitrosureas, as well as DNA damaging agents, which induce DNA damage when applied to a cell, and corticosteroid hormones.

Examples of alkylating agents include, inter alia, chloroambucil, cis-platinum, cyclodisone, flurodopan, methyl CCNU, piperazinedione, teroxirone. Topoisomerase I inhibitors encompass compounds such as camptothecin and camptothecin derivatives, as well as morpholinodoxorubicin. Doxorubicin, pyrazoloacridine, mitoxantrone, and rubidazone are illustrations of topoisomerase II inhibitors. RNA/DNA antimetabolites include L-alanosine, 5-fluoraouracil, aminopterin derivatives, methotrexate, and pyrazofurin; while the DNA antimetabolite group encompasses, for example, ara-C, guanozole, hydroxyurea, thiopurine. Typical antimitotic agents are colchicine, rhizoxin, taxol, and vinblastine sulfate. Other agents and factors include radiation and waves that induce DNA damage such as, γ-irradiation, X-rays, UV-irradiation, microwaves, electronic emissions, and the like. A variety of anti-cancer agents, also described as “chemotherapeutic agents,” function to induce DNA damage, all of which are intended to be of use in the combined treatment methods disclosed herein. Chemotherapeutic agents contemplated to be of use, include, e.g., adriamycin, bleomycin, 5-fluorouracil (5FU), etoposide (VP-16), camptothecin, actinomycin-D, mitomycin C, cisplatin (CDDP), podophyllotoxin, verapamil, and even hydrogen peroxide. The invention also encompasses the use of a combination of one or more DNA damaging agents, whether radiation-based or actual compounds, such as the use of X-rays with cisplatin or the use of cisplatin with troglitazone is contemplated. It is also contemplated that a combination of thiazolidinediones can be administered.

In treating pre-cancer or cancer according to the invention, one would contact the cells of a precancerous lesion or tumor cells with an agent in addition to the expression construct. This may be achieved by irradiating the localized tumor site with radiation such as X-rays, UV-light, γ-rays or even microwaves. Alternatively, the cells may be contacted with the agent by administering to the subject a therapeutically effective amount of a pharmaceutical composition comprising a compound such as, adriamycin, bleomycin, 5-fluorouracil, etoposide, camptothecin, actinomycin-D, mitomycin C, podophyllotoxin, verapamil, or more preferably, cisplatin. The agent may be prepared and used as a combined therapeutic composition, or kit, by combining it with troglitazone as described above.

Agents that directly cross-link nucleic acids, specifically DNA, are envisaged to facilitate DNA damage leading to a synergistic, anti-neoplastic combination with troglitazone. Cisplatinum agents such as cisplatin, and other DNA alkylating agents may be used. Cisplatin has been widely used to treat cancer, with efficacious doses used in clinical applications of 20 mg/m² for 5 days every three weeks for a total of three courses. Cisplatin is not absorbed orally and must therefore be delivered via injection intravenously, subcutaneously, intratumorally or intraperitoneally. Bleomycin and mitomycin C are other anticancer agents that are administered by injection intraveneously, subcutaneously, intratumorally or intraperitoneally. A typical dose of bleomycin is 10 mg/m², while such a dose for mitomycin C is 20 mg/m².

Agents that damage DNA also include compounds that interfere with DNA replication, mitosis and chromosomal segregation. Such chemotherapeutic compounds include adriamycin, also known as doxorubicin, etoposide, verapamil, podophyllotoxin, and the like. Widely used in a clinical setting for the treatment of neoplasms, these compounds are administered through bolus injections intravenously at doses ranging from 25-75 mg/m² at 21 day intervals for adriamycin, to 35-50 mg/m² for etoposide intravenously or double the intravenous dose orally.

Agents that disrupt the synthesis and fidelity of nucleic acid precursors and subunits also lead to DNA damage. As such a number of nucleic acid precursors have been developed. Particularly useful are agents that have undergone extensive testing and are readily available. As such, agents such as 5-fluorouracil (5-FU), are preferentially used by neoplastic tissue, making this agent particularly useful for targeting to neoplastic cells. Although quite toxic, 5-FU, is applicable in a wide range of carriers, including topical, however intravenous administration with doses ranging from 3 to 15 mg/kg/day being commonly used.

Other factors that cause DNA damage and have been used extensively include what are commonly known as γ-rays, X-rays, and/or the directed delivery of radioisotopes to tumor cells. Other forms of DNA damaging factors are also contemplated such as microwaves and UV-irradiation. It is most likely that all of these factors effect a broad range of damage DNA, on the precursors of DNA, the replication and repair of DNA, and the assembly and maintenance of chromosomes. Dosage ranges for X-rays range from daily doses of 50 to 200 roentgens for prolonged periods of time (3 to 4 weeks), to single doses of 2000 to 6000 roentgens. Dosage ranges for radioisotopes vary widely, and depend on the half-life of the isotope, the strength and type of radiation emitted, and the uptake by the neoplastic cells.

The skilled artisan is directed to “Remington's Pharmaceutical Sciences” 15th Edition, chapter 33, in particular pages 624-652. Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

In addition to combining troglitazone therapies with chemo- and radiotherapies, it also is contemplated that combination with other gene therapies will be advantageous. For example, implementing troglitazone in combination with the targeting of p53 or p16 mutations at the same time may produce an improved anti-cancer treatment. Any other tumor-related gene conceivably can be targeted in this manner, for example, p21, Rb, APC, DCC, NF-1, NF-2, BCRA2, p16, FHIT, WT-1, MEN-I, MEN-II, BRCA1, VHL, FCC, MCC, ras, myc, neu, raf, erb, src, fms, jun, trk ret, gsp, hst, bcl and abl. A therapeutic polynucleotide of the present invention, therefore, encompasses a polynucleotide that provides a medical advantage to the cell when administered. This could include, for example, a wild-type or dominant negative copy of any of the tumor-related genes previously listed.

It also should be pointed out that any of the foregoing therapies may prove useful by themselves. In this regard, reference to chemotherapeutics and gene therapy in combination also should be read as a contemplation that these approaches may be employed separately.

E. Formulations and Routes for Administration to Patients

Where clinical applications are contemplated, it will be necessary to prepare pharmaceutical compositions—expression vectors, virus stocks, proteins, antibodies and drugs—in a form appropriate for the intended application. Generally, this will entail preparing compositions that are essentially free of pyrogens, as well as other impurities that could be harmful to humans or animals.

One will generally desire to employ appropriate salts and buffers to render delivery vectors stable and allow for uptake by target cells. Buffers also will be employed when recombinant cells are introduced into a patient. Aqueous compositions of the present invention comprise an effective amount of the vector to cells, dissolved or dispersed in a pharmaceutically acceptable carrier or aqueous medium. Such compositions also are referred to as inocula. The phrase “pharmaceutically or pharmacologically acceptable” refers to molecular entities and compositions that do not produce adverse, allergic, or other untoward reactions when administered to an animal or a human. As used herein, “pharmaceutically acceptable carrier” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the vectors or cells of the present invention, its use in therapeutic compositions is contemplated. Supplementary active ingredients also can be incorporated into the compositions.

The inventors propose that local, regional delivery of troglitazone to a patient with cancer or pre-cancer conditions will be a very efficient method for delivering a therapeutically effective gene to counteract the clinical disease. Similarly, the chemo- or radiotherapy may be directed to a particular, affected region of the subject's body. Regional chemotherapy typically involves targeting anticancer agents to the region of the body where the cancer cells or tumor are located. Examples include intra-arterial, intracavity, intravesical, intrathecal, intrapleural, and intraperitoneal routes.

Intra-arterial administration is achieved using a catheter that is inserted into an artery to an organ or to an extremity. Typically, a pump is attached to the catheter. Intracavity administration describes when chemotherapeutic drugs are introduced directly into a body cavity such as intravesical (into the bladder), peritoneal (abdominal) cavity, or pleural (chest) cavity. Agents can be given directly via catheter. Intravesical chemotherapy involves a urinary catheter to provide drugs to the bladder, and is thus useful for the treatment of bladder cancer. Intrapleural administration is accomplished using large and small chest catheters, while a Tenkhoff catheter (a catheter specially designed for removing or adding large amounts of fluid from or into the peritoneum) or a catheter with an implanted port is used for intraperitoneal chemotherapy. Abdomen cancer may be treated this way. Because most drugs do not penetrate the blood/brain barrier, intrathecal chemotherapy is used to reach cancer cells in the central nervous system. To do this, drugs are administered directly into the cerebrospinal fluid. This method is useful to treat leukemia or cancers that have spread to the spinal cord or brain.

Alternatively, systemic delivery of the chemotherapeutic drugs may be appropriate in certain circumstances, for example, where extensive metastasis has occurred. Intravenous therapy can be implemented in a number of ways, such as by peripheral access or through a vascular access device (VAD). A VAD is a device that includes a catheter, which is placed into a large vein in the arm, chest, or neck. It can be used to administer several drugs simultaneously, for long-term treatment, for continuous infusion, and for drugs that are vesicants, which may produce serious injury to skin or muscle. Various types of vascular access devices are available.

The active compositions of the present invention may include classic pharmaceutical preparations. Administration of these compositions according to the present invention will be via any common route so long as the target tissue is available via that route. This includes oral, nasal, buccal, rectal, vaginal or topical. Alternatively, administration may be by orthotopic, intradermal, subcutaneous, intramuscular, intraperitoneal or intravenous injection. Such compositions would normally be administered as pharmaceutically acceptable compositions, described supra. The drugs and agents also may be administered parenterally or intraperitoneally. The term “parenteral” is generally used to refer to drugs given intravenously, intramuscularly, or subcutaneously.

Solutions of the active compounds as free base or pharmacologically acceptable salts can be prepared in water suitably mixed with a surfactant, such as hydroxypropylcellulose. Dispersions also can be prepared in glycerol, liquid polyethylene glycols, and mixtures thereof and in oils. Under ordinary conditions of storage and use, these preparations contain a preservative to prevent the growth of microorganisms.

The therapeutic compositions of the present invention are advantageously administered in the form of injectable compositions either as liquid solutions or suspensions; solid forms suitable for solution in, or suspension in, liquid prior to injection may also be prepared. These preparations also may be emulsified. A typical composition for such purpose comprises a pharmaceutically acceptable carrier. For instance, the composition may contain 10 mg, 25 mg, 50 mg or up to about 100 mg of human serum albumin per milliliter of phosphate buffered saline. Other pharmaceutically acceptable carriers include aqueous solutions, non-toxic excipients, including salts, preservatives, buffers and the like. Examples of non-aqueous solvents are propylene glycol, polyethylene glycol, vegetable oil and injectable organic esters such as ethyloleate. Aqueous carriers include water, alcoholic/aqueous solutions, saline solutions, parenteral vehicles such as sodium chloride, Ringer's dextrose, etc. Intravenous vehicles include fluid and nutrient replenishers. Preservatives include antimicrobial agents, anti-oxidants, chelating agents and inert gases. The pH, exact concentration of the various components, and the pharmaceutical composition are adjusted according to well known parameters. Suitable excipients for formulation with troglitazone include croscarmellose sodium, hydroxypropyl methylcellulose, iron oxides synthetic), magnesium stearate, microcrystalline cellulose, polyethylene glycol 400, polysorbate 80, povidone, silicon dioxide, titanium dioxide, and water (purified).

Additional formulations are suitable for oral adrninistration. Oral formulations include such typical excipients as, for example, pharmaceutical grades of mannitol, lactose, starch, magnesium stearate, sodium saccharine, cellulose, magnesium carbonate and the like. The compositions take the form of solutions, suspensions, tablets, pills, capsules, sustained release formulations or powders. When the route is topical, the form may be a cream, ointment, salve or spray.

An effective amount of the therapeutic agent is determined based on the intended goal, for example (i) inhibition of tumor cell proliferation or (ii) elimination of tumor cells. The term “unit dose” refers to physically discrete units suitable for use in a subject, each unit containing a predetermined-quantity of the therapeutic composition calculated to produce the desired responses, discussed above, in association with its administration, i.e., the appropriate route and treatment regimen. The quantity to be administered, both according to number of treatments and unit dose, depends on the subject to be treated, the state of the subject and the protection desired. Precise amounts of the therapeutic composition also depend on the judgment of the practitioner and are peculiar to each individual.

F. In Vitro, Ex Vivo, In Vivo Administration

As used herein, the term in vitro administration refers to manipulations performed on cells removed from an animal, including, but not limited to, cells in culture. The term ex vivo administration refers to cells that have been manipulated in vitro, and are subsequently administered to a living animal. The term in vivo administration includes all manipulations performed on cells within an animal.

In certain aspects of the present invention, the compositions may be administered either in vitro, ex vivo, or in vivo. U.S. Pat. Nos. 4,690,915 and 5,199,942, both incorporated herein by reference, disclose methods for ex vivo manipulation of blood mononuclear cells and bone marrow cells for use in therapeutic applications.

In vivo administration of the compositions of the present invention also is contemplated. Examples include, but are not limited to, chemotherapy of bladder epithelium by administration of the chemotherapeutic compositions of the present invention through intravesicle catheterization into the bladder (Bass et al., 1995), and chemotherapy of liver cells by infusion of appropriate chemotherapeutic compositions through the portal vein via a catheter (Bao et al., 1996). Additional examples include direct injection of tumors with the instant compositions, and either intranasal or intratracheal (Dong et al., 1996) instillation of chemotherapeutic compositions to effect transduction of lung cells.

G. Therapeutically Effective Amounts of Troglitazone and Other Thiazolidinediones

A therapeutically effective amount of a thiazolidinedione, such as troglitazone, that is combined with a second agent as treatment varies depending upon the host treated and the particular mode of administration. In one embodiment of the invention the dose range of a thiazolidinedione used will be about 0.5 mg/kg body weight to about 500 mg/kg body weight. The term “body weight” is applicable when an animal is being treated. When isolated cells are being treated, “body weight” as used herein should read to mean “total cell weight”. The term “total weight may be used to apply to both isolated cell and animal treatment. All concentrations and treatment levels are expressed as “body weight” or simply “kg” in this application are also considered to cover the analogous “total cell weight” and “total weight” concentrations. However, those of skill will recognize the utility of a variety of dosage range, for example, 1 mg/kg body weight to 450 mg/kg body weight, 2 mg/kg body weight to 400 mg/kg body weighty, 3 mg/kg body weight to 350 mg/kg body weighty, 4 mg/kg body weight to 300 mg/kg body weight, 5 mg/kg body weight to 250 mg/kg body weighty, 6 mg/kg body weight to 200 mg/kg body weight, 7 mg/kg body weight to 150 mg/kg body weighty, 8 mg/kg body weight to 100 mg/kg body weight, or 9 mg/kg body weight to 50 mg/kg body weight. Further, those of skill will recognize that a variety of different dosage levels will be of use, for example, 1 mg/kg, 2 mg/kg, 3 mg/kg, 4 mg/kg, 5 mg/kg, 7.5 mg/kg, 10, mg/kg, 12.5 mg/kg, 15 mg/kg, 17.5 mg/kg, 20 mg/kg, 25 mg/kg, 30 mg/kg, 35 mg/kg, 40 mg/kg, 45 mg/kg, 50 mg/kg, 60 mg/kg, 70 mg/kg, 80 mg/kg, 90 mg/kg, 100 mg/kg, 120 mg/kg, 140 mg/kg, 150 mg/kg, 160 mg/kg, 180 mg/kg, 200 mg/kg, 225 mg/kg, 250 mg/kg, 275 mg/kg, 300 mg/kg, 325 mg/kg, 350 mg/kg, 375 mg/kg, 400 mg/kg, 450 mg/kg, 500 mg/kg, 550 mg/kg, 600 mg/kg, 700 mg/kg, 750 mg/kg, 800 mg/kg, 900 mg/kg, 1000 mg/kg, 1250 mg/kg, 1500 mg/kg, 1750 mg/kg, 2000 mg/kg, 2500 mg/kg, and/or 3000 mg/kg. Of course, all of these dosages are exemplary, and any dosage in-between these points is also expected to be of use in the invention. Any of the above dosage ranges or dosage levels may be employed for troglitazone or other thiazolidinediones alone or for such a compound in combination with an anti-cancer drug.

“Therapeutically effective amounts” are those amounts effective to produce beneficial results, particularly with respect to cancer treatment, in the recipient animal or patient. Such amounts may be initially determined by reviewing the published literature, by conducting in vitro tests or by conducting metabolic studies in healthy experimental animals. Before use in a clinical setting, it may be beneficial to conduct confirmatory studies in an animal model, preferably a widely accepted animal model of the particular disease to be treated. Preferred animal models for use in certain embodiments are rodent models, which are preferred because they are economical to use and, particularly, because the results gained are widely accepted as predictive of clinical value.

As is well known in the art, a specific dose level of active compounds such as troglitazone and thiazolidinediones compounds for any particular patient depends upon a variety of factors including the activity of the specific compound employed, the age, body weight, general health, sex, diet, time of administration, route of administration, rate of excretion, drug combination, and the severity of the particular disease undergoing therapy. The person responsible for administration will determine the appropriate dose for the individual subject. Moreover, for human administration, preparations should meet sterility, pyrogenicity, general safety and purity standards as required by FDA Office of Biologics standards.

In some embodiments, the troglitazone or thiazolidinediones compound will be administered in combination with a second agent. So long as a dose of second agent that does not exceed previously quoted toxicity levels is not required, the effective amounts of the second agents may simply be defined as those amounts effective to reduce the cancer growth when administered to an animal in combination with the troglitazone agents. This is easily determined by monitoring the animal or patient and measuring those physical and biochemical parameters of health and disease that are indicative of the success of a given treatment. Such methods are routine in animal testing and clinical practice.

Chemotherapy is typically administered in regular cycles. A cycle may involve one dose, after which several days or weeks without treatment ensues for normal tissues to recover from the drug's side effects. Doses may be given several days in a row, or every other day for several days, followed by a period of rest. If more than one drug is used, the treatment plan will specify how often and exactly when each drug should be given. The number of cycles a person receives may be determined before treatment starts (based on the type and stage of cancer) or may be flexible, in order to take into account how quickly the tumor is shrinking. Certain serious side effects may also require doctors to adjust chemotherapy plans to allow the patient time to recover.

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### EXAMPLE 1

### Inhibition of Cell Proliferation of Osteosarcoma Cells

I. Materials and Methods

Cell Culture and Treatment Regimen

Saos-2 cells were purchased from ATCC (Rockville, Md.) and cultured in 10% fetal bovine serum, penicillin/streptomycin, and DMEM media. Cells exposed to a single treatment of troglitazone, 5-fluorouracil, and/or cis-platinum for 3 days. At the end of 3 days media was tested for lactate dehydrogenase (LDH) activity as a measure of cell toxicity and DNA content was determined as a measure of cell proliferation. The phrase “cell proliferation” is used herein interchangeably with “cell growth.”

Transfection and Luciferase Assay

Cells were transfected using the calcium phosphate method of transfection. 10 μg of AOX-PPRE-tk-CATreporter plasmid was transfected into Saos-2 cells. Twenty-four hours after transfection, cells were treated for 24 hours in the presence or absence of troglitazone or 9-cis retinoic acid (SIGMA™, St. Louis, Mo.). Cells lysates were prepared and CAT activity was determined by ELISA as described by Boehringer-Mannheim.

Lactate dehydrogenase activity assay (LDH) Assay

Media from treated cells was assayed for LDH activity with a colorimetric dye-oxidation method which has been previously described (11). Briefly, the assay mix including lactate, NAD, INT dye and diaphorase in PBS was added to media from the cells and incubated 30 minutes. Red color development is indicative of INT oxidation to formazan product, giving an indication of LDH activity when measured at 490 nm.

DNA Assay

Concentration of cellular DNA was determined using Hoechst dye H 33258 and a Hoefer DyNA Quant fluorometer according to manufacturer's instructions. Results are expressed as μg DNA/well. All experiments were done in triplicate.

[³H]thymidine Incorporation

Saos-2 cells (10,000/well) were seeded into wells of 12-well plates and grown for 4 days until near confluent. The cells were then incubated for 16 h with 5% FBS/DMEM and antibiotics alone or in the presence of troglitazone. [Methyl-³H]thymidine (1 μCi) and thymidine (3 μM) were then added to each well, and the incubation was continued for another 4 h. Cells were rinsed with ice-cold PBS twice and were treated with ice-cold 10% TCA. The cells were then rinsed twice with PBS and solubilized in 400 μl of solution composed of 0.03% SDS in 0.3 N NaOH. The extracts were neutralized with HCl, and radioactivity incorporated into the cells was quantified by liquid scintillation spectrometry.

Ribonuclease Protection Assay

To determine whether troglitazone effected any of the cell cycle regulator genes, ribonuclease protection assay (RPA) was performed measuring mRNAs encoding p130, Rb, p107, p53, p57, p27, p21, p19, p18, p16, p14/15 and the housekeeping gene products L32 and GAPDH. Cells were in log phase in 5% FBS/DMEM in the presence or absence of 5 μg/ml troglitazone for 24 h. Cells were lysed and total RNA was isolated.

Antisense transcripts were labeled by in vitro transcription with α-[³²P]UTP (800 Ci/mmol) and T7 RNA polymerase. Ribonuclease protection assay was performed using kit according to manufacturer instructions (PharMingen, San Diego, Calif.). Conditions that were optimized included using 20 μg of total RNA from Hs578T cells and a 1:200 dilution of RNAse A/T (200 units/ml RNase A and 10,000 units/ml RNase T). Protected fragments were isolated on a 5% acrylamide, 8M urea denaturing gel, dried, and exposed on both x-ray film and phosphoimager (Molecular Dynamics). Results are reported as the ratio of hOTR to cyclophilin signal. All experiments were done in triplicate from different RNA preparations.

Northern Analysis for PPAR γmRNA

Total cellular RNAs from control and troglitazone treated cells were isolated (12). Total RNA (20 μg/sample) was fractionated on a 1% formaldehyde agarose gel and transferred onto a nylon filter (Hybond-N, Amersham Life Science, Arlington, Ill.). Northern hybridization was performed using Quikhyb hybridization solution according to manufacturer's recommendations (Stratagene, La Jolla, Calif.). Quantitation of each band was performed using phosphoimager (Molecular Dynamics). The cDNA for mouse PPAR γ was a gift from Dr. Steven Kliewer (Glaxo Wellcome Research and Development, Research Triangle Park, N.C.).

Western Analysis for PPAR γ and p21 Protein

Aliquots of samples with same amount of protein, determined using Bradford assay (Bio-Rad, Hercules, Calif.), were mixed with loading buffer [final concentrations of 62.5 mM Tris-HCl (pH 6.8), 2.3% SDS, 100 mM dithiothreitol, and 0.005% bromophenol blue], boiled, fractionated in a 12% SDS-PAGE, and transferred onto a 0.45-μm nitrocellulose membrane (Bio-Rad). The filters were blocked with 2% fat-free milk in PBS and probed with either anti-PPAR γ (1:1000 dilution) or anti-p21 antibodies (1:2000 dilution), (Santa Cruz Biotechnology, Santa Cruz, Calif.) in PBS containing 0.1% Tween 20 (PBST) and 1% fat-free milk. The membranes were washed once in PBST and incubated with horseradish peroxidase-conjugated goat antirabbit secondary antibody (Bio-Rad) in PBST containing 1% fat-free milk. After washing four times in PBST, the membranes were visualized using the enhanced chemiluminescence (ECL) western blotting detection system (Amersham Life Science).

II. Effect of Troglitazone on Osteosarcoma Cells

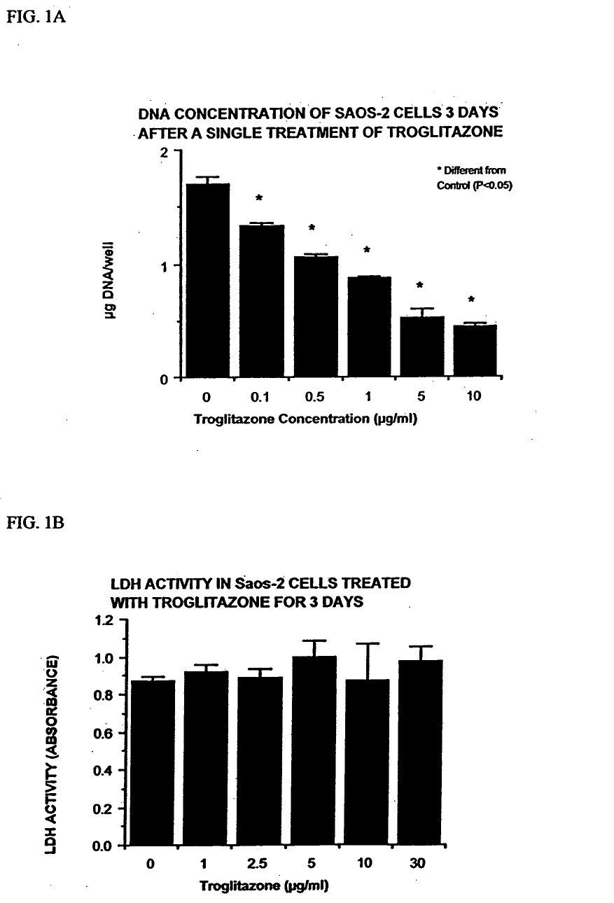

FIG. 1A shows DNA content per well following a single treatment with troglitazone. Cells were exposed for 3 days and cell number determined by measuring DNA content. Cells were exposed for 3 days and cell number determined by measuring DNA content. The IC₅₀ for troglitazone is between about 1 μg/ml (2.27 μM) and about 10 μg/ml. Thus, single treatment oftroglitazone demonstrates a dose dependent decrease in cell proliferation 3 days following treatment of Saos-2 cells.

FIG. 1B shows lactate dehydrogenase (LDH) levels measured in the media after 3 days of treatment. LDH levels are indicative of cell death either through apoptosis or necrosis. LDH levels are not altered at any concentration of troglitazone used, as compared to controls, indicating that troglitazone, while not being toxic to cells, inhibits cell proliferation. Thus, up to 30 μg/ml troglitazone is not lethal to Saos-2 cells as shown in FIG. 1B with no increase of lactate dehydrogenase (LDH) activity as compared to control in the media of cells treated for 3 days with troglitazone. Moreover, cells incubated in the presence of ³H-thymidine to access the affect on DNA/RNA synthesis and cell proliferation demonstrates a dose dependent decrease in thymidine incorporation. Approximately a 45% decrease in 3H-thymidine incorporation is demonstrated after 24 h exposure to 10 μg/ml troglitazone. These results indicate that troglitazone regulates cell proliferation by inhibition of some factor(s) that regulate the cell cycle.

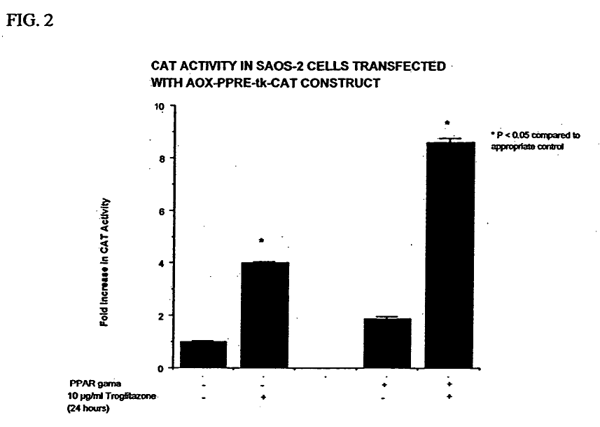

Examination of the mechanism of action of troglitazone was performed by using the reporter construct AOX—PPRE-tk-CAT. This expression vector contains a DNA sequence that specifically binds PPAR-γ thereby activating chloramphenicol acetyltransferase (CAT) expression. FIG. 2 shows troglitazone treatment to increase CAT expression 4-fold (lane 2 vs lane 1) in the absence of transfected PPAR-Y as well as the presence of 0.67 μg transfected PPAR γ(lanes 3 and 4) thus first demonstrating the existence of functional PPARγ in Saos-2 cells.

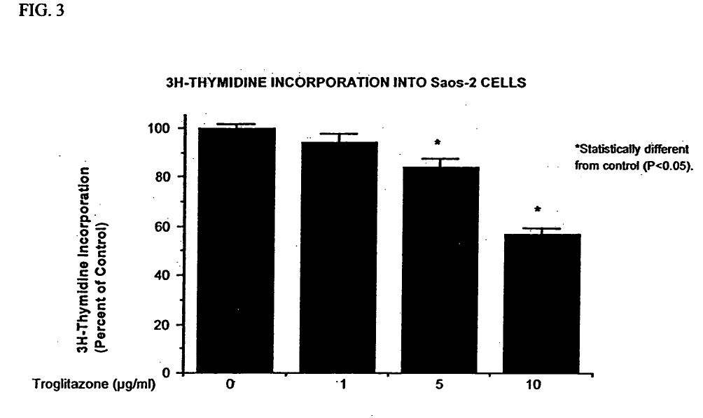

³H uridine incorporation was used to determine whether troglitazone inhibited cell cycle progression. Cells (80% confluent) were treated with troglitazone for 18 hours and examined for 3H-uridine uptake into cell lysates. As shown in FIG. 3, a dose of 5 μg/ml troglitazone caused a 16% inhibition of ³H-uridine uptake while a 10 μg/ml dose caused a 43% inhibition.

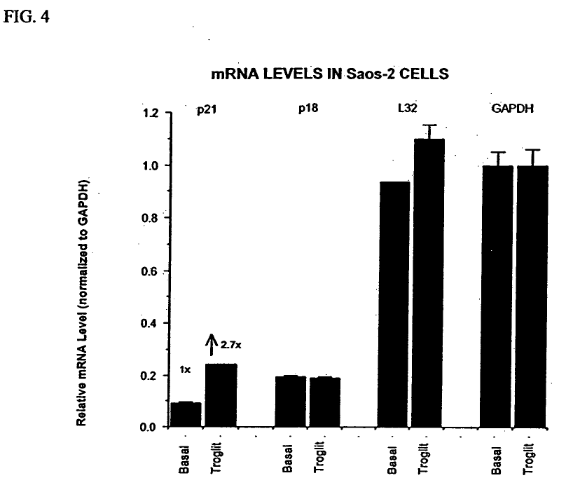

FIG. 4 shows the effect upon mRNA levels for cell cycle inhibitor genes in response to a single treatment of 1 μM troglitazone for 24 hr, which revealed a 2.6-fold increase in p21 mRNA levels. Levels were normalized to GAPDH. Another control used is L32 which is seen to change very little. p18 is shown not change. Other cell cycle inhibitor genes demonstrating little change in response to troglitazone treatment include p130, retinoblastoma, p107, p27, p16, and −15.

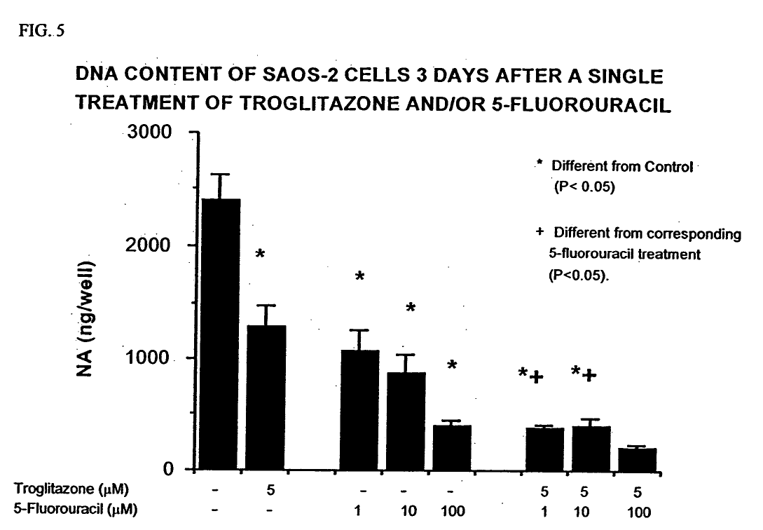

FIG. 5 shows the effects of toxic chemotherapeutic agent, 5-fluorouracil (5-FU), revealing that combinatorial therapy with 5 μM troglitazone and 1 μM 5-FU was equivalent to 100 μM dose of 5-FU alone. Combinatorial therapy reduces the dose of 5-FU needed by a factor of 100.

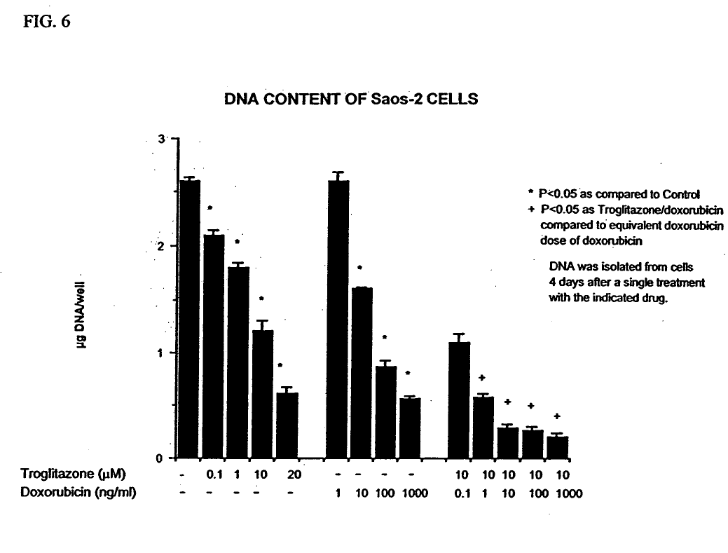

In FIG. 6, troglitazone demonstrates a similar lowering of the dose of doxorubicin needed to inhibit cell proliferation when comparing doxorubicin treatment alone and co-administering doxorubicin with 10 μM troglitazone.

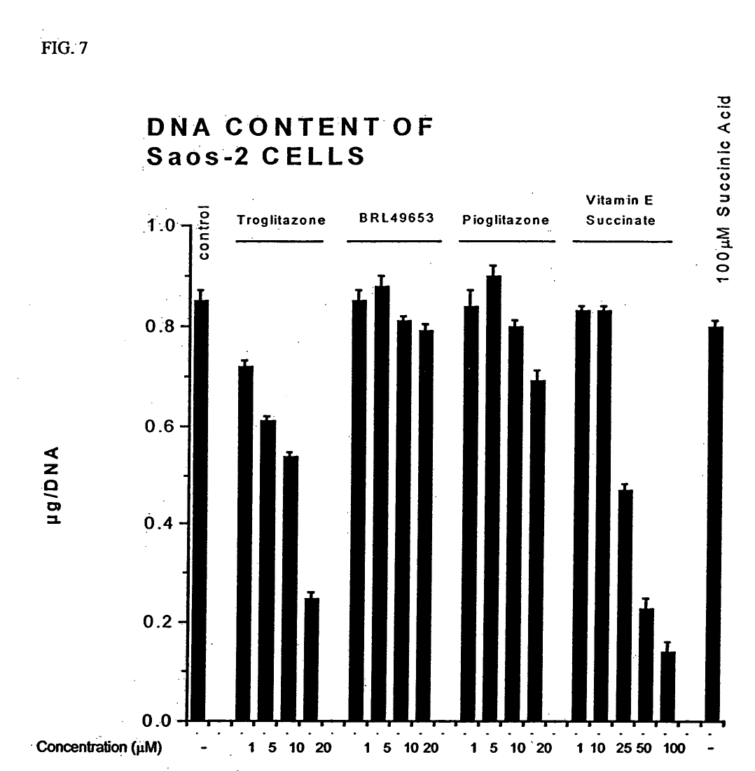

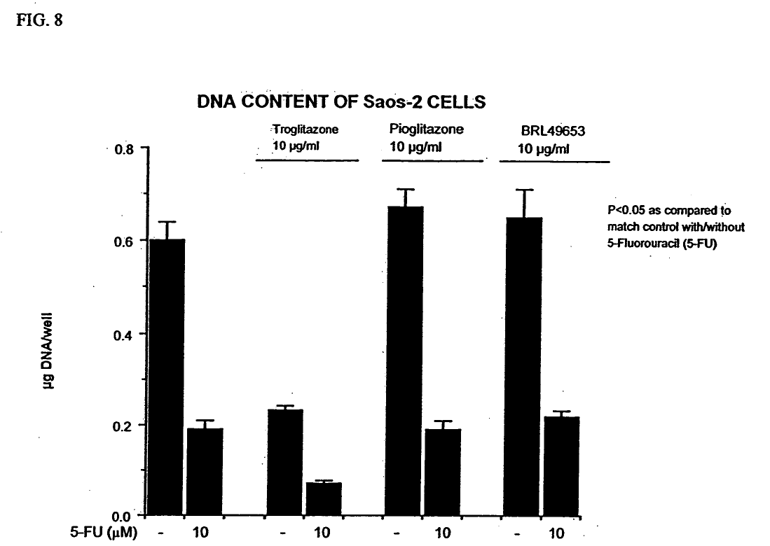

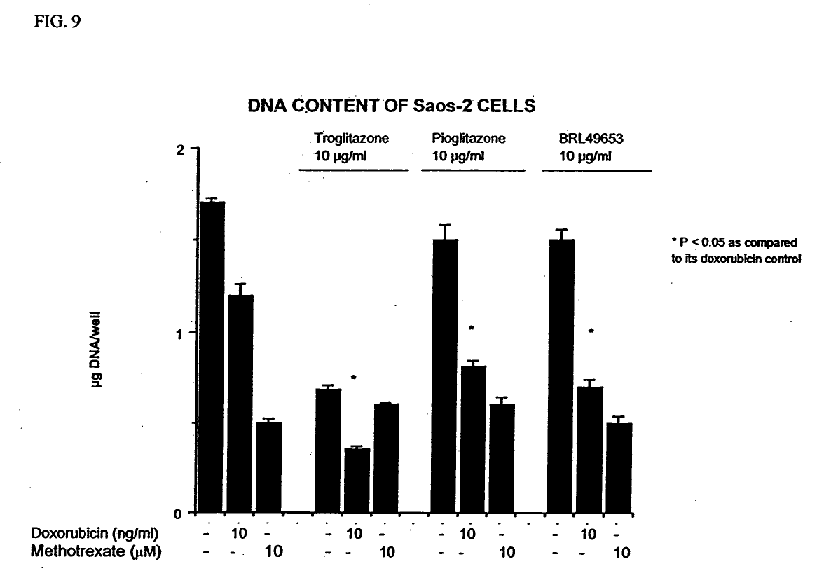

Examining compounds similar to troglitazone for inhibitory activity upon cell proliferation should further delineate the mechanism of action of troglitazone. The thiazolidinediones, pioglitazone and BRL49653 have a 10-100 fold greater affinity for PPAR-γ but do not contain the vitamin E moiety in their structures. Comparing these compounds as well as vitamin E succinate revealed that troglitazone was much more potent in inhibiting cell proliferation than the other thiozolidinediones after a single treatment (FIG. 7). Vitamin E succinate demonstrates a dose responsive inhibition of cell proliferation somewhat less effective than troglitazone. At 100 μM vitamin E, cells are being killed as evidenced by microscopic examination and LDH levels in the media. This effect appears specific since 100 μM succinic acid had no effect upon cell proliferation (last column of FIG. 7). The data from FIG. 7 are indicative that troglitazone may be acting additionally through its vitamin E properties in addition to its ability to bind to PPAR-γ. Further comparisons with the thiazolidinediones in combinatorial therapy reveal that troglitazone is superior to pioglitazone and BRL49653 in its ability to inhibit cell proliferation. The results of combinatorial therapy with 5-FU are depicted in FIG. 8 and those of doxorubicin are depicted in FIG. 9. As also seen in FIG. 9, none of the thiazolidinediones had any additional inhibitory effect on cell proliferation when administered with methotrexate.

Taken together, this data demonstrate that functional PPAR-γ exist in human osteosarcoma, Saos-2, cells. This is also the first demonstration of PPAR-γ mRNA expression is normal osteoblasts. However, thiozolidinediones with a higher affinity for PPAR-γ were less effective than troglitazone in inhibiting cell proliferation suggesting that troglitazone may work through multiple mechanisms. However, higher doses of vitamin E were needed to cause similar growth inhibitory responses when compared to troglitazone. Thus, it is plausible that troglitazone utilizes both functions in inhibiting cell proliferation. Troglitazone because of its unique properties of causing terminal differentiation and low toxicity may be an excellent candidate for prophylactic therapy for individuals at high risk for cancer.

### EXAMPLE 3

### Inhibition of Cell Proliferation in Human Renal Cell Carcinomas

I. Materials and Methods

Cell Culture and Treatment Regimen

Human renal tumor cells (URMC 3, 6, 7) were cultured in 5% fetal bovine serum, penicillin/streptomycin, and alpha-MEM media. Cells exposed to a single treatment of troglitazone, 5-fluorouracil, and/or doxorubicin for 3 days. At the end of 3 days DNA content was determined as a measure of cell proliferation.

Transfection and Luciferase Assay

A tandem PPRE luciferase expression plasmid was constructed using three copies of a consensus PPRE 5′ to the luciferase gene. Cells were transfected using the calcium phosphate method of transfection. 10 μg of PPRE/luciferase reporter plasmid was transfected into URMC 3, 6, and 7 cells. A CMV-CAT reporter plasmid was cotransfected (0.5 μg) with PPRE/luciferase as an internal control for transfection efficiency. CAT activity was measured by ELISA as described by Boehringer-Mannheim. Twenty-four hours after transfection, cells were treated for 24 hours in the presence or absence of troglitazone or 9-cis retinoic acid (Sigma). Cells lysates were prepared and luciferase activity was determined using an AutoLumat luminometer.

DNA Assay

Concentration of cellular DNA was determined using Hoechst dye H 33258 and a Hoefer DyNA Quant fluorometer according to manufacturer's instructions. Results are expressed as μg DNA/well. All experiments were done in triplicate.

II. Results

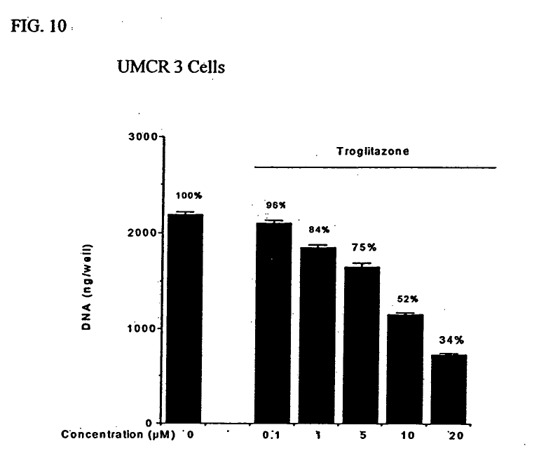

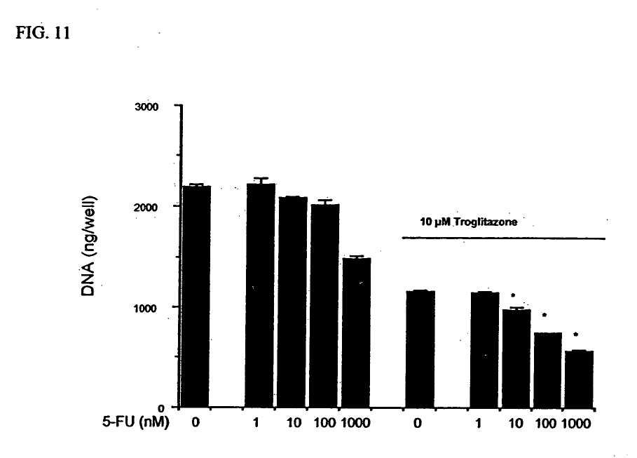

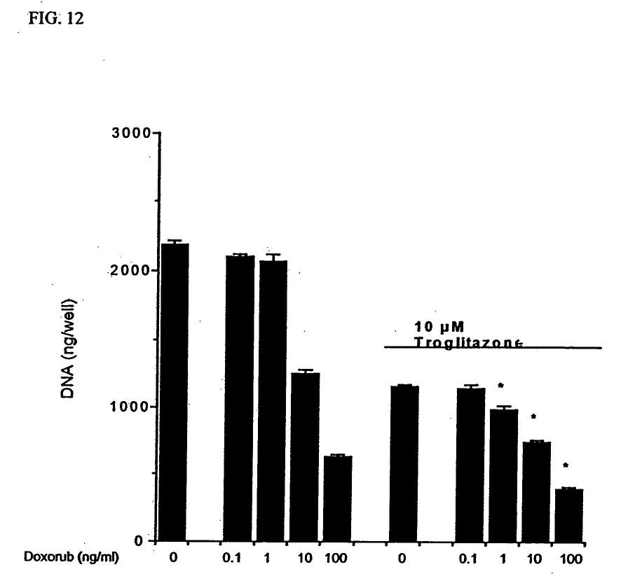

In FIG. 10, troglitazone causes a dose responsive inhibition of cell proliferation in the UMCR 3 renal cell carcinoma cell line. The IC₅₀ is between 5 and 10 μM. Combinatorial therapy with troglitazone and 5-fluorouracil (5-FU) is more effective in inhibiting cell proliferation in UMCR 3 cells as compared to that of troglitazone or 5-FU treatment alone (FIG. 11). Similarly, in FIG. 12, combinatorial treatment of UMCR 3 cells with troglitazone with doxorubicin is more effective in inhibiting cell proliferation than either treatment alone. Combinatorial therapy of these agents could prove effective treatment in renal cell carcinoma.

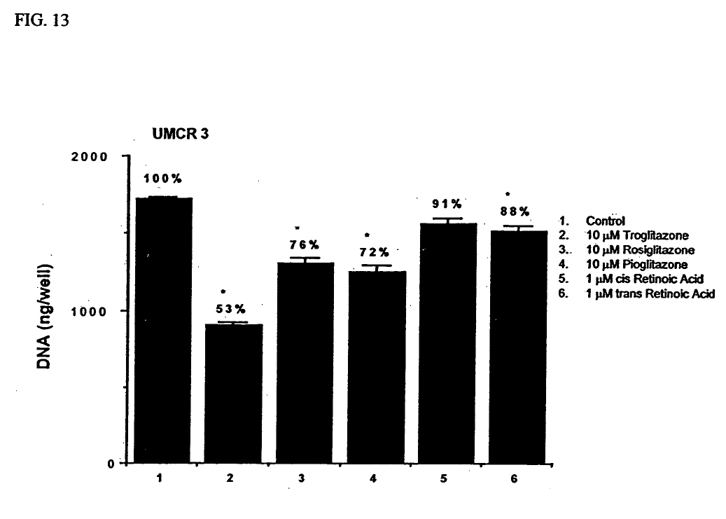

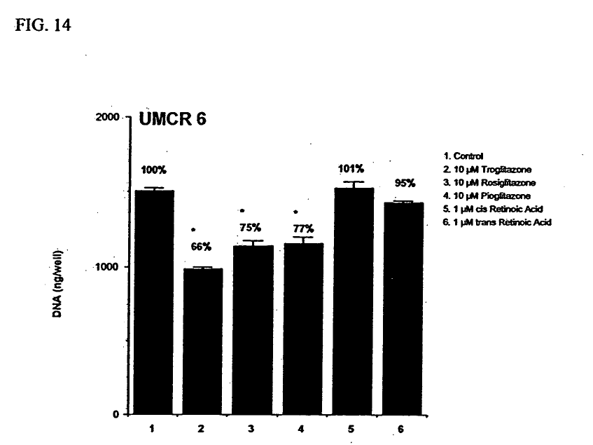

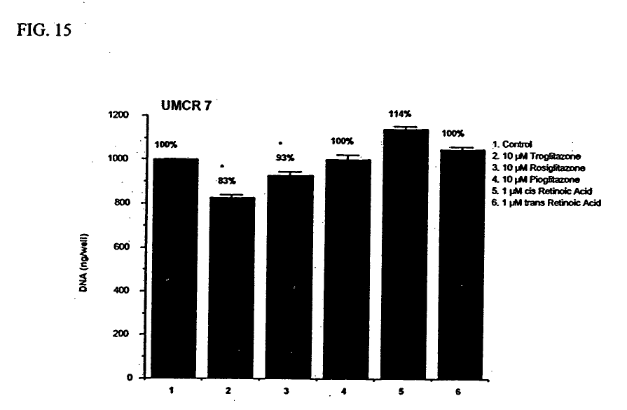

In type 2 diabetes, the antidiabetic potency of these compounds is related to their affinity to the transcriptional factor, PPARγ. Rosiglitazone (BRL49653) has the highest affinity (40 nM) for PPARy with pioglitazone and troglitazone having a 5- and 100-fold lower affinity as compared to rosiglitazone. Thus, three renal cell carcinoma cell lines were used to compare the growth inhibitory effects of these three thiazolidinediones to determine whether a similar rank order of potency occurred. In FIGS. 13-15, a 10 μM concentration of troglitazone, rosiglitazone, and pioglitazone were compared. As well, 9-cis-retinoic acid and all trans retinoic acids were used as controls since these compounds are also known to cause terminal differentiation in some cell types and also bind to retinoic acid X receptor which heterodimerizes with PPARγ to activate transcription. The retinoic acids demonstrate little on no growth inhibitory activity in these cells. However, the thiazolidinediones inhibit cell proliferation to different degrees, with troglitazone being the most potent of the three. This suggests that other mechanisms other than activation of PPARγ may play a role in inhibition of cell proliferation.

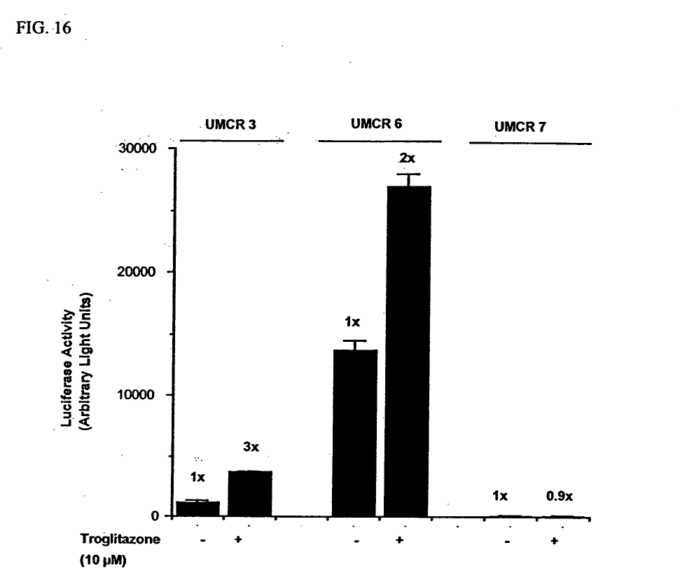

In FIG. 16, the three cell lines were examined for functional PPARγ by transiently transfecting the cells with a PPRE response element linked to a luciferase reporter gene. UMCR 3 cells had a low basal PPARγ activity and 10 μM troglitazone stimulated transcription 3-fold. UMCR 6 cells demonstrate a 6-fold higher basal activity as compared to UMCR 3 cells and troglitazone stimulated activity another 2-fold. UMCR 7 cells demonstrate little or no PPARγ-mediated transcription either basally or in response to troglitazone treatment. This indicates that UMCR 7 cells have no functional PPARγ. These cells were much less responsive to growth inhibition to the three thiazolidinediones as compared to that of UMCR 3 and 6 cells. These combined data indicate that growth inhibition could be through PPARγ-mediated as well as independent pathways.

### EXAMPLE 4

### Inhibition of Proliferation of Ovarian Cancer Cells

I. Materials and Methods

Cell Culture and Treatment Regimen

Human ovarian tumor cells were cultured in 5% fetal bovine serum, penicillin/streptomycin, and alpha-MEM media Cells exposed to a single treatment of troglitazone, taxol, and/or cisplatin for 3 days. At the end of 3 days DNA content was determined as a measure of cell proliferation.

DNA Assay

Concentration of cellular DNA was determined using Hoechst dye H 33258 and a Hoefer DyNA Quant fluorometer according to manufacturer's instructions. Results are expressed as μg DNA/well. All experiments were done in triplicate.

II. Results

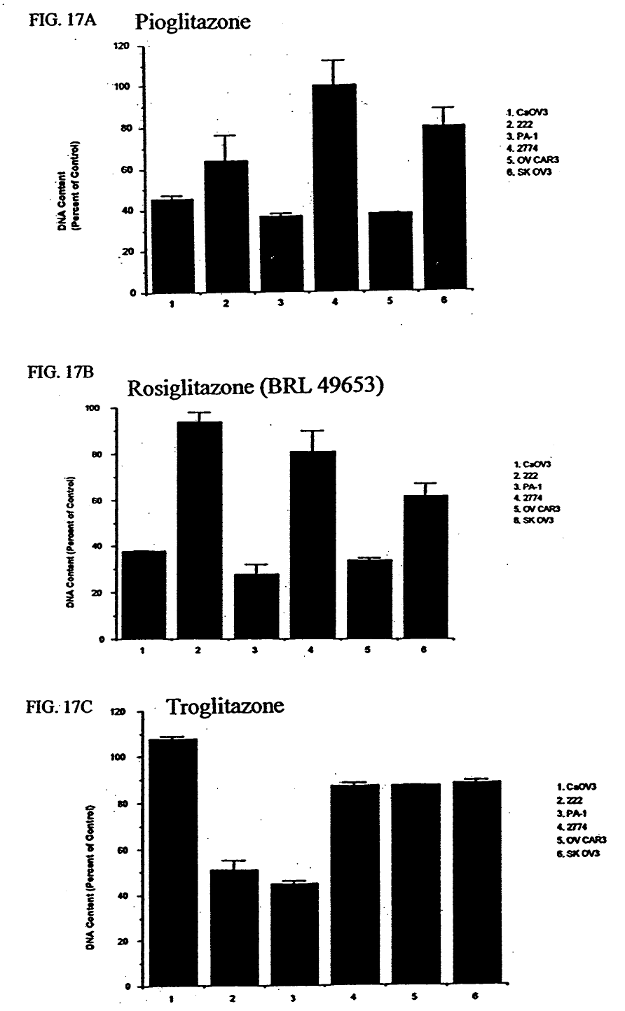

In FIG. 17, inhibition of cell proliferation by a 10 μM dose of pioglitazone, rosiglitazone, or troglitazone are compared in six human ovarian tumor cell lines. Each respective cell line demonstrated different responses with respect to the thiazolidinedione used. For example, pioglitazone and rosiglitazone inhibited cell proliferation by 60% in CaOV3 cells with troglitazone demonstrating no inhibitory activity. However, in 222 cells, troglitazone inhibited growth by 50% while pioglitazone inhibited by 36% and rosiglitazone inhibited by 4%. PA-1 cells were similarly inhibited by 60-70% by all three compounds. These data indicate that responsiveness to an individual thiazolidinedione can not be readily predicted.

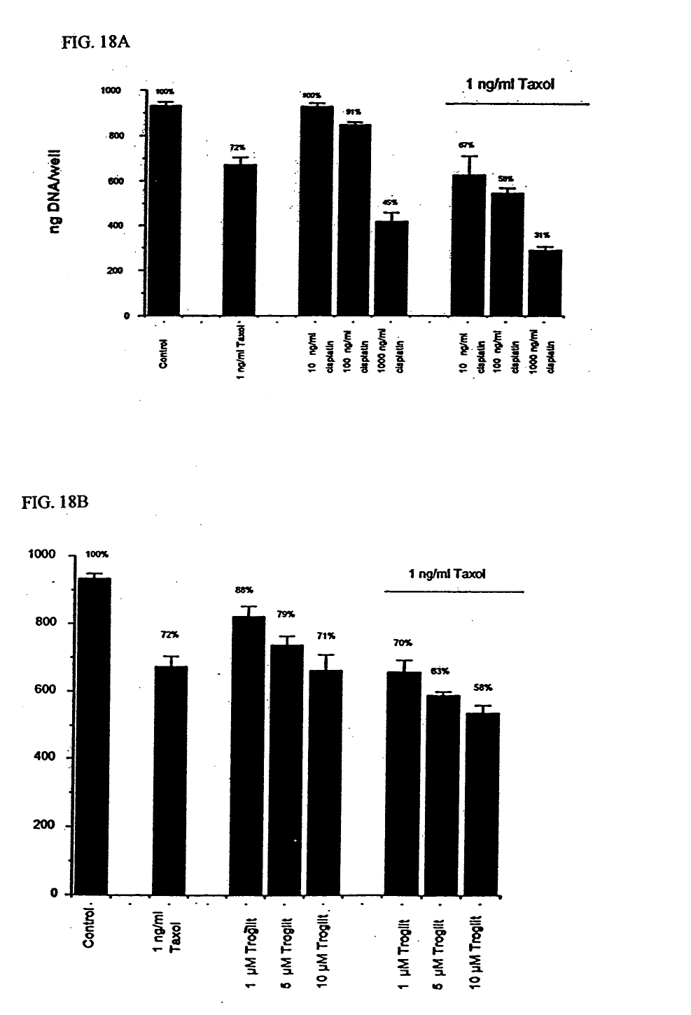

In FIG. 18, combinatorial therapy was used to determine whether thiazolidinediones would enhance inhibition of cell proliferation in human ovarian tumor cells. Taxol, which has been demonstrated to be initially effective against ovarian cancer, was used in combination with troglitazone. Combinatorial treatment using 1 ng/ml of taxol with either 5 or 10 μM troglitazone enhanced inhibitory activity by about 12% (42%) over that of either agent used alone (FIG. 18A). This regimen is not as effective as that already in use, combinatorial therapy of taxol:cisplatin (FIG. 18B). A 70% inhibition of cell proliferation occurs with 100 ng/ml cis-platin: 1 ng/ml taxol as compared to that of taxol alone (28%) or cis-platin (55%) alone.

### EXAMPLE 5

### Human Treatment with Thiazolidinedione Compounds Either Alone or in Combination with Other Treatments

As a type 2 diabetic drug, troglitazone is administered orally at doses up to 600 mg/day. Blood concentrations approach 7 μM in patients treated at 600 mg/day. These are levels comparable to those seen in cell culture to inhibit tumor cell proliferation. Thus, troglitazone could be given orally on a daily basis in different regimens depending upon the combinatorial therapy being administered. Alternatively, as in the case of ovarian cancer that predominantly metastasizes within the abdominal cavity, troglitazone could be injected in a carrier solution interabdominally.

Troglitazone and other thiazolidinedione compounds could be given 2 months prior to administering standard chemotherapeutic agents. This would have the effect of creating elevated blood levels of thiazolidinedione and should shrink the tumor. Lower doses of the standard chemotherapeutic agents would then be administered at levels one-fourth, one-tenth, one-fiftieth, and one-one hundredth of the current standard doses used. Inhibition of tumor growth would determine whether lower doses of a standard chemotherapeutic agent(s) could be more effective in combination with troglitazone or other thiazolidinediones.

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and methods in the steps or in the sequence of steps of the method described herein without departing from the concept, spirit and scope of the invention. More specifically, it will be apparent that certain agents that are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.

U.S. Pat. No. 4,690,915

U.S. Pat. No. 5,199,942

U.S. Pat. No. 5,478,852

Auersperg et al., Am. J. Obstetr. GynecoL, 173:558-565, 1995.

Auersperg et al., Lab. Invest. 71:510-518, 1994.

Bao et al., Hum. Gene Ther., 7:355-365, 1996.

Bass et al., Cancer Gene Ther., 2:97-104, 1995.

Chinery et al., Nature Medicine 3:1233-1241, 1997.

Chomczynski et al., Analytic Biochem. 162:156, 1987.

Culver et al., Science, 256:1550-1552, 1992.

Dong et al., Hum. Gene Ther., 7:319-331, 1996.

Dyck et al., Int J Cancer 69:429436, 1996.

Jacobs et al., J Natl Cancer Inst 84:1793-1798, 1992.

Kliewer et al., Nature 358:771-774, 1992.

Kubota et al., Cancer Res 58:3344-3352, 1998.

Mok et al., Oncogene 16:2381-2387, 1998.

Mok et al., Cancer Res 52:5119-5122. 1993.

Mosmann, J. Immunol. Methods, 65:55-63, 1983.

Mueller et al., Molecular Cell 1:465470, 1998.

Piver et al., Gynecology Oncology, 2nd Edn. New York: McGraw Hill Inc., pp.250-291, 1992.

Qazi and McGuire, CA Cancer J Clin 45:88-101, 1995.

Remington's Pharmaceutical Sciences 15th Edition, chapter 61.

Rubinstein et al., J. Natl. Cancer Inst., 82:1113-1120, 1990.

Sarraf et al., Nature Medicine 4:1046-1052, 1998.

Sears et al., Mol. Cell Biol. 16:3410-3418, 1996.

Sherr, Science, 274:1672-1677, 1996.

Smith and Rutledge, Natl. Cancer Inst Monogr., 42:141-143, 1975.

Tontonoz et al., Nucleic Acids Res 22:5628-5634, 1994.

Tontonoz et al., Genes Dev 8:1224-1234, 1994.

Tontonoz et al., Mol. Cell Biol. 15:351-357, 1995.

Tontonoz et al., Proc Natl Acad Sci USA 94:237-241, 1997.

Tsao et al., Gynecol Oncology 48:5-10, 1993.

Warrel et al., N Engl. J Med 329:177-189, 1993.

Young et al., N. Engl. J. Med., 299:1261-1266, 1978.

1-38. (canceled)

39. A method for inhibiting the growth of a cancer cell comprising: contacting the cancer cell with a thiazolidinedione compound and a DNA damaging agent, in amounts effective to inhibit the growth of the cancer cell.

40. The method of claim 39, wherein the DNA damaging agent is a chemotherapeutic drug or radiation.

41. The method of claim 40, wherein the DNA damaging agent is radiation.

42. The method of claim 39, wherein the thiazolidinedione compound is a troglitazone.

43. The method of claim 39, wherein the thiazolidinedione compound is a pioglitazone.

44. The method of claim 39, wherein the thiazolidinedione compound is a rosiglitazone.

45. The method of claim 39, wherein the cancer cell is a mammalian cancer cell.

46. The method of claim 45, wherein the cancer cell is a human cancer cell.

47. The method of claim 39, wherein the contacting occurs in vitro.

48. The method of claim 39, wherein the contacting occurs in vivo.

49. The method of claim 39, wherein the cancer cell is selected from a group consisting of a bladder, blood, bone, bone marrow, brain, breast, colon, esophagus, gastrointestine, head, kidney, liver, lung, nasopharynx, neck, ovary, prostate, skin, stomach, and uterus cell.

50. The method of claim 49, wherein the cancer cell expresses PPAR-γ.

51. The method of claim 49, wherein the cancer cell is a bone cancer cell.

52. The method of claim 51, wherein the bone cancer cell is an osteosarcoma cell.

53. The method of claim 51, wherein the cancer cell is a precursor to osteosarcoma.

54. The method of claim 49, wherein the cancer cell is an ovarian cancer cell.

55. The method of claim 49, wherein the cancer cell is a renal cancer cell.

56. The method of claim 40, wherein the cancer cell is contacted with a chemotherapeutic drug.

57. The method of claim 56, wherein the chemotherapeutic drug comprises an alkylating agent, mitotic inhibitor, antibiotic, nitrosurea, antimetabolite, corticosteroid hormone, or other antineoplastic agent.

58. The method of claim 57, wherein the chemotherapeutic drug comprises an alkylating agent.

59. The method of claim 57, wherein the chemotherapeutic drug comprises a mitotic inhibitor.

60. The method of claim 57, wherein the chemotherapeutic drug comprises an antibiotic.

61. The method of claim 57, wherein the chemotherapeutic drug comprises a nitrosourea.

62. The method of claim 57, wherein the chemotherapeutic drug comprises an antimetabolite.

63. The method of claim 57, wherein the chemotherapeutic drug comprises a corticosteroid hormone.

64. The method of claim 57, wherein the chemotherapeutic drug comprises an antineoplastic agent.

65. The method of claim 39, wherein the thiazolidinedione compound is contacted with a cancer cell by administering the thiazolidinedione regionally, endoscopically, intravenously, intralesionally, percutaneously, subcutaneously, intraperitoneally, intratracheally, intramuscularly, or by perfusion.

66. The method of claim 57, wherein the thiazolidinedione and the chemotherapeutic drug are suitably dispersed in a pharmacologically acceptable formulation.

67. The method of claim 56, wherein the thiazolidinedione compound is contacted with the cancer cell at the same time as contact with the chemotherapeutic agent.

68. The method of claim 67, wherein the thiazolidinedione and the chemotherapeutic agent are combined in a therapeutic formulation.

69. The method of claim 39, wherein the cancer cell is a tumor cell in a tumor.

70. The method of claim 69, further comprising resecting the tumor.

71. The method of claim 41, wherein the cancer cell is irradiated with X-ray irradiation, UV-irradiation, γ-irradiation, or microwaves.

72. The method of claim 71, wherein the thiazolidinedione compound is contacted with the cancer cell at the same time as irradiation.

73. The method of claim 39, further comprising contacting the cancer cell with a therapeutic polynucleotide selected from the group consisting of a Dp gene, p21, p16, p27, E2F, Rb, APC, DC, NF-1, NF-2, WT-1, MEN-I, MEN-II, BRCA1, VHL, FCC, MCC, ras, myc, neu, raf, erb, src, fins, jun, trk, ret, gsp, hst, bcl, abl, Bax, Bcl-Xs and E1A; wherein the therapeutic polynucleotide is expressed in the cancer cell.

74. A method for treating cancer in a patient comprising administering to the patient troglitazone and a chemotherapeutic drug in amounts effective to produce a therapeutic benefit.

75. A method for inhibiting the cell cycle progression of a mammalian cancer cell comprising contacting the cell with troglitazone and a chemotherapeutic drug in amounts effective to inhibit the cell cycle progression of the cell.

76. A method of treating cancer in a patient comprising administering to the patient troglitazone and a chemotherapeutic drug in amounts that are therapeutically effective.

77. A method for treating microscopic residual cancer comprising the steps of:
(i) identifying a patient having a resectable tumor;
(ii) resecting said tumor; and
(iii) contacting the tumor bed with troglitazone and a chemotherapeutic drug in amounts effective to produce a therapeutic benefit.

78. A method for treating a subject having a tumor comprising the steps of:
(i) surgically revealing said tumor; and
(ii) contacting said tumor with troglitazone and a chemotherapeutic drug in amounts effective to produce a therapeutic benefit.

79. A method for treating a subject having a tumor comprising the step of perfusing said tumor, over an extended period of time, with troglitazone and a chemotherapeutic drug in amounts effective to produce a therapeutic benefit.

80. A method for inhibiting the growth of a cancer cell comprising:
i) contacting the cancer cell with a composition comprising troglitazone; and
ii) contacting the cancer cell with a chemotherapeutic agent or irradiating the cancer cell, in amounts effective to inhibit growth of the cancer cell.

81. The method of claim 80, wherein the cancer cell is contacted with a chemotherapeutic agent.

82. The method of claim 81, wherein the composition comprises troglitazone and a chemotherapeutic agent.

83. The method of claim 80, wherein the cancer cell is a bone cancer cell.

84. The method of claim 83, wherein the bone cancer cell is an osteosarcoma cell.

85. The method of claim 80, wherein the cancer cell is an ovarian cancer cell.

86. The method of claim 80, wherein the cancer cell is a renal cancer cell.
